(19) United States
(12) Patent Application Publication
(10) Pub. No.: US 2021/0393857 A1
(43) Pub. Date: Dec. 23, 2021

(54) SMALL MOLECULE DRUGS AND METHODS TO ACCELERATE OSSEOINTEGRATION

(71) Applicant: The Regents of the University of California, Oakland, CA (US)

(72) Inventors: Ichiro NISHIMURA, Venice, CA (US); Akishige HOKUGO, Los Angeles, CA (US); Kenzo MORINAGA, Fukuoka (JP); Hodaka SASAKI, Saitama (JP)

(73) Assignee: The Regents of the University of California, Oakland, CA (US)

(21) Appl. No.: 17/289,470

(22) PCT Filed: Nov. 4, 2019
(86) PCT No.: PCT/US19/59693

§ 371 Date: Apr. 28, 2021

Related U.S. Application Data
(60) Provisional application No. 62/755,698, filed on Nov. 5, 2018.

(51) Int. Cl. A61L 27/54; A61K 31/522; A61K 31/366; A61K 31/437; A61K 31/4709; A61K 31/4745; A61K 31/197; A61L 27/06; A61K 6/84

(52) U.S. Cl. CPC A61L 27/54; A61K 31/522; A61K 31/366; A61K 31/437; A61K 31/4709; A61L 2300/216; A61K 31/197; A61L 27/06; A61K 6/84; A61L 2430/02; A61L 2430/12; A61K 31/4745

(57) ABSTRACT

Methods for enhancing or accelerating osseointegration of an implant into bone marrow of a subject, the methods comprising increasing expression of peripheral clock neuronal PAS domain protein 2 (NPAS2) in the bone marrow, are provided. Expression of NPAS2 is increased by administration of a Npas2 modulating compound to the subject.

### CROSS-REFERENCE TO RELATED APPLICATIONS

This application claims the benefit of and priority to U.S. Provisional Application No. 62/755,698, filed Nov. 5, 2018, the contents of which are incorporated herein by reference in its entirety.

### FIELD OF THE INVENTION

The invention relates to methods for enhancing or accelerating osseointegration of an implant into bone marrow of a subject, the method comprising increasing expression of peripheral clock neuronal PAS domain protein 2 (NPAS2) in the bone marrow.

### BACKGROUND OF THE INVENTION

Titanium (Ti)-based biomaterials have been widely used in implantable medical devices for orthopedic and dental applications. In a classic study, Laing et al. (1967) examined the tissue reaction of metallic materials implanted in rabbit muscle and concluded that commercially pure Ti and Ti alloys were among those which induced the least foreign body reaction and minimal fibrosis. The biologically inert characteristics of Ti-implants initially explained the establishment of direct bone to implant connection or osseointegration without a layer of soft tissue encapsulation. Surface functionalization of Ti implants has been shown to improve and accelerate the osseointegration process, which includes moderately rough surface topography and nanotopography, increasing surface energy and incorporating bone inductive biological molecules. The outcome of these functionalization technologies suggest that Ti-biomaterials with a complex surface can induce biological reactions leading to the establishment of enhanced osseointegration, although the underlying mechanisms are not yet fully understood. Several reports from experimental animal studies and human cases suggest that sound vitamin D level in the host is a critical factor for the establishment of osseointegration. While it is well established that activated vitamin D in the kidney controls calcium ions in the circulation; its precise action in bone tissue is less understood. The impaired osseointegration in vitamin D deficient rats was not due to the lack of bone formation around implant but rather driven by the decreased bone bonding to the implant surface.

Accordingly, there remains a need for methods and effective therapeutic compositions for enhancing os seointegration.

To determine the role of vitamin D in implant osseointegration, we previously performed a whole genome microarray study of peri-implant tissue derived from vitamin D sufficient and deficient rats. This study unexpectedly identified the molecular circadian clock gene Neuronal PAS domain protein 2 (Npas2) as highly associated with the successful development of osseointegration. Npas2 is a transcription factor containing a basic helix-loop-helix (bHLH) structure for DNA binding. Due to a sequence similarity with Circadian Locomoter Output Cycles Kaput (Clock), Npas2 has been considered a member of circadian rhythm regulatory molecules. Circadian rhythms are found in many processes throughout the body and are centrally regulated by a timing system found in the hypothalamic suprachiasmatic nucleus (SCN). On a molecular level, the core clock molecules Brain and Muscle Arnt-like protein 1 (Bmal1) and Clock dimerize to participate in a transcription/translation feedback loop involving Period (Per) and Cryptochrome (Cry) genes. The circadian rhythm is also found in peripheral tissues including bone. Recently, Ti disc with a complex surface was found to upregulate Npas2 expression of bone marrow stromal cells (BMSC) in vitro. The weighed gene co-expression network analysis revealed that the upregulation of Npas2 driven by the Ti implant was selective and not seen in other circadian rhythm-related genes, suggesting that an independent molecular mechanism was responsible for the increased Npas2 expression in response to Ti-biomaterials.

Taken together, the mechanistic role of Npas2 in implant osseointegration has been proposed.

### SUMMARY OF THE INVENTION

In one aspect, the invention provides a method for enhancing osseointegration of an implant into bone marrow of a subject, the method comprising increasing expression of peripheral clock neuronal PAS domain protein 2 (NPAS2) in the bone marrow.

In another aspect, the invention provides a method for accelerating osseointegration of an implant into bone marrow of a subject, the method comprising increasing expression of peripheral clock neuronal PAS domain protein 2 (NPAS2) in the bone marrow.

In still another aspect, the invention provides a method for re-establishing an implant-bone integration in a subject, the method comprising increasing expression of peripheral clock neuronal PAS domain protein 2 (NPAS2) in the bone marrow.

In another aspect, the invention provides a method for improving or accelerating osseointegration of an implant into bone marrow of a subject, the method comprising administering to the subject a pharmaceutical composition comprising a NPAS2 polypeptide or a Npas2 modulating compound, wherein the Npas2 modulating compound increases expression of peripheral clock neuronal PAS domain protein 2 (NPAS2) in the bone marrow.

In a further aspect, the invention provides a method for improving or accelerating bone repair and wound healing, the method comprising administering to the subject a pharmaceutical composition comprising a NPAS2 polypeptide or a Npas2 modulating compound, wherein the Npas2 modulating compound increases expression of peripheral clock neuronal PAS domain protein 2 (NPAS2) in bone marrow and wound tissue.

Other features and advantages will become apparent from the following detailed description, examples, and figures. It should be understood, however, that the detailed description and the specific examples while indicating certain embodiments of the invention are given by way of illustration only, since various changes and modifications within the spirit and scope of the invention will become apparent to those skilled in the art from this detailed description.

### BRIEF DESCRIPTION OF THE FIGURES

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings.

FIGS. 1A-1E illustrate the effect of Ti biomaterials on circadian clock gene expression in BMSC. FIG. 1A shows the surface characterization of commercially pure Ti discs treated as machined or sand blasted and acid etched (SLA). FIG. 1B shows the surface topography evaluation of machined and SLA Ti discs by optical interferometric profilometry. FIG. 1C shows the surface roughness measurements (n=3 per group). FIG. 1D graphically illustrates the time course expression of clock genes by human BMSC cultured on polystyrene culture plates, machined Ti discs and SLA Ti discs (n=6 per time point in each group). NPAS2 expression was significantly increased when BMSCs were cultured on SLA Ti discs. *: p<0.05. FIG. 1E illustrates the detrended harmonious regression analysis of clock gene expression data. Compared with control BMSC on polystyrene, SLA Ti disc attenuated and/or modified circadian expression pattern.

FIGS. 2A-2H illustrate the development of a mouse implant osseointegration model. FIG. 2A shows the experimental implants (0.6 mm in diameter and 10 mm in length) that were designed with a handle. After insertion into an osteotomy site created in the femur bone marrow, the Ti rod was clipped to generate a 4-mm long implant. FIG. 2B shows the surface of the experimental implant that was treated as SLA or machined, respectively. FIG. 2C shows a 4-mm implant placed in the femur bone marrow at a depth of 4 mm from the distal femur joint surface to avoid contact with the epiphyseal cartilage and growth plate. FIG. 2D shows how at predetermined times, mouse femurs were harvested and trimmed to expose the edge of the implant. The mechanical withholding strength of the implant was measured by the implant pushout test. The breakpoint load (N) was determined as the implant push-out value. FIG. 2E graphically shows the implant push-out value increased over the healing time until 3 wk and then reached a plateau. The graph shows the mean push-out value ±SEM at 1, 2, 3, 4 and 8 wk (n=3, 4, 4, 4 and 4, respectively). FIG. 2F graphically shows a separate experiment, in which harvested mouse femurs were fixed in 10% buffered formalin and processed for nondecalcified epoxy resin-embedded histological sections. The Goldner trichrome-stained sections were used to determine the bone-implant contract (BIC) ratio. The BIC ratio increased over the healing time until 3 wk. The graph shows the mean BIC ratio ±SEM at 1, 2, 3, 4 and 8 wk (n=4, 4, 4, 4 and 4, respectively). FIG. 2G shows a representative EDS elemental analysis of a 3-wk implant recovered after the push-out test. The remnant tissue contained O, P and Ca elements, whereas the Ti element was decreased. FIG. 2H graphically shows the analysis by EDS of the entire surface of recovered implant, and the calculated average elemental weight % of Ti, Ca and P for each implant. The Ti weight % progressively decreased over the healing time until 3 wk and then reached a plateau. Ti elemental analysis by EDS mirrored the reverse trend of the BIC ratio, but the measurement variation was small. The graph shows the mean EDS element contents (weight %) ±SEM at 1, 2, 3, 4 and 8 wk (n=3, 4, 4, 4 and 4, respectively).

FIGS. 3A-3D FIGS. 3A-3D illustrate femur bone characterization of wild type (WT), Npas2+/− and Npas2−/− mice. FIG. 3A is a diagram of Npas2 allele and genomic DNA PCR. Due to replacement of exon 3 (E3) encoding basic helix-loop-helix (bHLH) sequence with a LacZ expression reporter cassette (LacZ/Neo), the mutant Npas2 protein lacked the DNA binding function. FIG. 3B shows the size and shape of femurs (n=3 per group) from 15-wk-old WT, Npas2+/− and Npas2−/− mice were indistinguishable. FIG. 3C shows the micro-CT three-dimensional trabecular bone structure of femurs. FIG. 3D shows no significant differences were observed in quantitative analysis of femur trabecular bone structure (n=6 per group) that was evaluated by microCT.

FIGS. 4A-4G illustrate impaired osseointegration in Npas2+/− and Npas2−/− mice. FIG. 4A shows that after 3 wks of recovery, the implant push-out values of SLA implants placed in Npas2+/− (n=8) and Npas2−/− (n=4) mice were significantly smaller than that of WT mice (n=12). The implant push-out value of machined implants in WT, Npas2+/− and Npas2−/− mice did not show a significant difference. The graph shows the mean±SEM. FIG. 4B are nondecalcified histological sections showing bone formation around the implant and bone-implant contact, which appeared to be unaffected by Npas2+/− and Npas2−/− mutation. Due to variations among sections, quantitative measurements were not performed. FIG. 4C show representative SEM observation of SLA implants after the implant push-out test. The plate-like remnant tissue covered the surface of SLA implants recovered from WT mice. SLA implants recovered from Npas2+/− and Npas2−/− mice were associated with the reticular remnant tissue (*) and clear exposure of Ti implant surface (arrows). FIG. 4D illustrates that EDS elemental analysis revealed larger Ti exposure of SLA implants recovered from Npas2+/− (n=8) and Npas2−/− (n=4) mice than in those recovered from WT mice (n=12). The mean±SEM. *: p<0.05 against WT control. FIG. 4E shows the EDS analysis of Ca and P of the remnant tissue on SLA implants and the femur cortical bone of WT, Npas2+/− and Npas2−/− mice. FIG. 4F plots the tissue coverage area on SLA implant that was estimated from the Ti elemental analysis and correlated with the implant push-out value. In WT mice, the estimated tissue coverage area and the implant push-out value was positively correlated, whereas these values did not correlate in Npas2+/− and Npas2−/− mice. FIG. 4G show high-magnification SEM that revealed dense collagen fibers in the remnant tissue of SLA implants recovered from WT mice, whereas collagen fiber structures were not clearly observed in the remnant tissues of Npas2+/− and Npas2−/− mice.

FIGS. 5A-5E illustrate an unbiased chemical genetics analysis that was used to determine the molecular mechanisms underlying implant osseointegration. FIG. 5A is a flow diagram of chemical genetics analysis using BMSC carrying Npas2-LacZ reporter system. FIG. 5B illustrates high throughput screening of LOPAC®¹²⁸⁰ compounds for Npas2-LacZ expression of mouse BMSC. Hit compounds were identified as z-score >2.5 or <−2.5. FIG. 5C shows the validation of Npas2-LacZ expression of hit compounds in triplicated experiments. The compounds (black bars) significantly modulated the Npas2-LacZ expression (p<0.05) compared to the untreated control (white bar) were identified. FIG. 5D are diagrammatic comparisons of the neuroskeletal pathway to a proposed altered neuroskeletal pathway. Neurotransmitter-induced β2 adrenergic receptor in BMSC has been shown to regulate bone remodeling as described as neuroskeletal regulation. The pharmacological actions of identified compounds clustered in the down regulation of cAMP and α2 adrenergic receptors. An altered neuroskeletal regulation was proposed as a molecular mechanism of osseointegration. FIG. 5E graphs the results of human BMSC exposed to Ti discs (machined and SLA) that were re-analyzed for the expression of adrenergic receptors. Compared to the conventional culture condition on polystyrene plate, BMSC exposed to SLA Ti disc exhibited significantly upregulated expression of α2 and β1 adrenergic receptors; but not of β2 adrenergic receptor.

FIGS. 6A-6B illustrate the outcome of an in vivo experiment to show that methyldopa improves implant osseointegration in the early healing stage. FIG. 6A shows that the push-out value of femur implants in mice with B-DAE-DCD surface were increased at week 2 (W2) and further increased at week 3 (W3). FIG. 6B shows, using machined surface (smooth surface) or B-DAE-DCD surface (rough surface) implants in femurs of mice, that daily intraperitoneal (IP) injections of methyldopa had greater push-out value than vehicle-treated control mice.

### DETAILED DESCRIPTION OF THE INVENTION

The present subject matter may be understood more readily by reference to the following detailed description which forms a part of this disclosure. It is to be understood that this invention is not limited to the specific products, methods, conditions or parameters described and/or shown herein, and that the terminology used herein is for the purpose of describing particular embodiments by way of example only and is not intended to be limiting of the claimed invention.

Unless otherwise defined herein, scientific and technical terms used in connection with the present application shall have the meanings that are commonly understood by those of ordinary skill in the art. Further, unless otherwise required by context, singular terms shall include pluralities and plural terms shall include the singular.

As employed above and throughout the disclosure, the following terms and abbreviations, unless otherwise indicated, shall be understood to have the following meanings.

In the present disclosure the singular forms “a,” “an,” and “the” include the plural reference, and reference to a particular numerical value includes at least that particular value, unless the context clearly indicates otherwise. Thus, for example, a reference to “a compound” is a reference to one or more of such compounds and equivalents thereof known to those skilled in the art, and so forth. The term “plurality”, as used herein, means more than one. When a range of values is expressed, another embodiment includes from the one particular and/or to the other particular value. Similarly, when values are expressed as approximations, by use of the antecedent “about,” it is understood that the particular value forms another embodiment. All ranges are inclusive and combinable.

As used herein, the terms “component,” “composition,” “composition of compounds,” “compound,” “drug,” “pharmacologically active agent,” “active agent,” “bioactive agent” “bioactive drug”, “therapeutic,” “therapy,” “treatment,” or “medicament” are used interchangeably herein to refer to a compound or compounds or composition of matter which, when administered to a subject (human or animal) induces a desired pharmacological and/or physiologic effect by local and/or systemic action.

As used herein, the terms “treatment” or “therapy” (as well as different forms thereof) include preventative (e.g., prophylactic), curative or palliative treatment. As used herein, the term “treating” includes alleviating or reducing at least one adverse or negative effect or symptom of a condition, disease or disorder.

“Administration” to a subject is not limited to any particular delivery system and may include, without limitation, parenteral (including subcutaneous, intravenous, intramedullary, intraarticular, intramuscular, or intraperitoneal injection).

The compositions of the invention may be administered topically or parenterally (e.g., intravenous, subcutaneous, intraperitoneal, and intramuscular). Further, the compositions of the invention may be administered by intravenous infusion or injection. The composition of the invention may be administered by intramuscular or subcutaneous injection. In some embodiments, the composition of the invention may be administered surgically. As used herein, a “composition” or “pharmaceutical composition” refers to any composition that contains a “pharmaceutically effective amount” or “therapeutically effective amount” (these terms are used interchangeably herein) of one or more active ingredients (e.g., a Npas2 modulating compound). A “therapeutically effective amount” refers to an amount effective, at dosages and for periods of time necessary, to achieve the desired therapeutic result. A therapeutically effective amount of a molecule may vary according to factors such as the disease state, age, sex, and weight of the individual, and the ability of the molecule to elicit a desired response in the individual. A therapeutically effective amount is also one in which any toxic or detrimental effects of the molecule are outweighed by the therapeutically beneficial effects. The composition, when administered to a subject (human or animal) induces a desired pharmacological and/or physiologic effect by local and/or systemic action.

The terms “subject,” “individual,” and “patient” are used interchangeably herein, and refer to an animal, for example a human, to whom treatment, including prophylactic treatment, with the pharmaceutical composition according to the present invention, is provided. The term “subject” as used herein refers to human and non-human animals. The terms “non-human animals” and “non-human mammals” are used interchangeably herein and include all vertebrates, e.g., mammals, such as non-human primates, (particularly higher primates), sheep, dog, rodent, (e.g. mouse or rat), guinea pig, goat, pig, cat, rabbits, cows, horses and non-mammals such as reptiles, amphibians, chickens, and turkeys.

In one aspect, the invention provides a method for enhancing or accelerating osseointegration of an implant into bone marrow of a subject, the method comprising increasing expression of peripheral clock neuronal PAS domain protein 2 (NPAS2) in the bone marrow.

The term “osseointegration” is defined herein as a direct structural and functional connection between bone and implant surface without a formation/attachment of fibrous tissue between the implant and bone, and without encapsulation by such soft/fibrous tissue.

In some embodiments, expression of NPAS2 is increased by administration of a Npas2 modulating compound to the subject.

As used herein a “Npas2 modulating compound” is a compound (or a plurality of compounds) or a composition of matter (e.g., drug(s)) which, when administered to a subject produces or stimulates a desired pharmacological and/or physiologic effect by local and/or systemic action, and in particular, the Npas2 modulating compound effects an upregulation (increase) of genetic expression of the Npas2 gene to thereby increase synthesis of the functional gene product, peripheral clock neuronal PAS domain protein 2 (NPAS2) in cells exposed to or stimulated by the compound, for example in human bone marrow stromal cells (BMSC).

### Npas2 Modulating Compounds

### A1 Adenosine Receptor Antagonists

In some embodiments, the adenosine receptor antagonist that may be used in accordance with embodiments described herein has a selectivity for A1 adenosine receptors over A2 adenosine receptors (also called an “A₁-selective antagonist”) and decreases beta adrenergic receptor-triggered cAMP signaling. In certain embodiments, the adenosine receptor antagonist is 8-(p-sulfophenyl) theophylline has the following chemical structure:

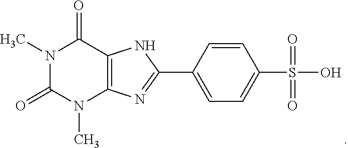 [SMILES: Cn1c(=O)c2[nH]c(-c3ccc(S(=O)(=O)O)cc3)nc2n(C)c1=O ; "."]

In additional embodiments, 1,3-dialkylxanthines, such as 1,3-dialkyl-8-(p-sulfophenyl)xanthines, may be used in accordance with embodiments described herein. In an embodiment, 1,3-dipropyl-8-phenylxanthine represents a potent and somewhat selective A1-receptor antagonist about 23-fold more potent at A1 receptors than at A2 receptors (Daly, J W, et al., J Med Chem. 1985; 28(4):487-492, which is incorporated herein by reference in its entirety). According to Daly, a p-hydroxyaryl substituent further enhances potency of the 1,3-dipropyl-8-phenylxanthine at both adenosine A1 and A2 receptors. In some embodiments, the 8-(2-amino-4-chlorophenyl)-1,3-dipropylxanthine, which is a very potent and selective antagonist for A1 receptors, being nearly 400-fold more potent at adenosine A1 than at A2 receptors, may be used in accordance with embodiments described herein. In additional embodiments, the adenosine receptor antagonist having a selectivity for A1 adenosine receptors over A2 adenosine receptors that may be used in accordance with embodiments described herein is 1-isoamyl-3-isobutylxanthine, which has the following chemical structure:

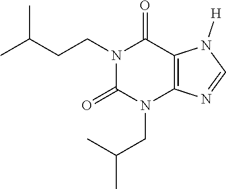 [SMILES: CC(C)CCn1c(=O)c2[nH]cnc2n(CC(C)C)c1=O]

In some embodiments, 8-substituted 1,3-dipropylxanthines may be used in accordance with embodiments described herein. In certain embodiments, 8-substituted 1,3-dipropylxanthines may be the A1-receptor antagonists used in accordance with embodiments described herein, including but not limited to (R)-3,7-dihydro-8-(1-methyl-2-phenylethyl)-1,3-dipropyl-1H-purine-2,6-dione, which was found to be a potent compound at the A1 receptor, however, (R)-3,7-dihydro-8-(1-phenylpropyl)-1,3-dipropyl-1H-purine-2,6-dione was found to be more selective at the A1 adenosine receptor (Peet, et al., J Med Chem. 1993 Dec. 10; 36(25):4015-20, which is incorporated herein by reference in its entirety).

The introduction of 8-cycloalkyl groups instead of 8-aryl groups is favorable for affinity to the A1 adenosine receptor; in an embodiment, a cycloalkylxanthine derivative, such as DPCPX (1,3-dipropyl-8-cyclopentylxanythine) or also called CPX (however, CPX is 8-Cyclopentyl-1,3-dimethylxanthine, also an A1 adenosine receptor antagonist), may be used in accordance with embodiments described herein (Muller and Jacobson, Handb Exp Pharmacol. Author manuscript; available in PMC 2014 Jan. 7; Muller and Jacobson, Biochim Biophys Acta. 2011 May; 1808(5): 1290-1308; and Auchampach, et al., JPET 308:846-856, 2004, which are incorporated herein by reference in their entirety). DPCPX has the following chemical structure:

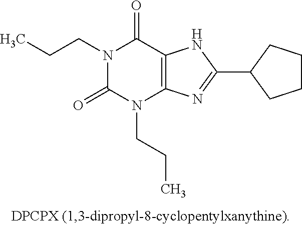 [SMILES: CCCn1c(=O)c2[nH]c(C3CCCC3)nc2n(CCC)c1=O]

In additional embodiments, the adenosine A1 receptor antagonists selective over A2 adenosine receptors may have bulky cycloalkyl substituents in the xanthine 8-position, such as 3-noradamantyl (e.g., rolofylline, i.e., 1,3-dipropyl-8-(3-noradamantyl)xanthine (also called “KW3902”) and 1-butyl-3-(3-hydroxypropyl)-8-(3-noradamantyl)xanthine (also called “PSB—36”)), (substituted) norbornyl (naxifylline, i.e., 1,3-dipropyl-8-[2-(5,6-epoxynorbonyl)]-xanthine (also called “BG-9719”, and “CVT124”), and the lactone, i.e., norbornyllactone-substituted xanthines), dicyclopropylmethyl (also called “MPDX” and “KF15372”) and 1,3-dipropyl-8-[1-(4-propionate)-bicyclo-[2,2,2]octyl]xanthine (i.e., toponafylline, also called “B G-9928”), which are very potent and selective A₁ antagonists; analogs of these A1 adenosine receptor antagonists, including but not limited to the 2-thio analog of DPCPX, 2-thio-DPCPX, and replacement of the 3-propyl residue of DPCPX by a phenyl, benzyl or chiral methylbenzyl residue, and a DPCPX analogs, including those having in the 3-position polar hydroxy groups, such as the hydroxylated DPCPX derivative PSB-16, or PSB-16 converted to its phosphoric acid ester disodium salt, also may be used in accordance with embodiments described herein (Muller and Jacobson, Handb Exp Pharmacol. Author manuscript; available in PMC 2014 Jan. 7, and Auchampach, et al., JPET 308:846-856, 2004, which are incorporated herein by reference in their entirety).

Rolofylline has the following chemical structure:

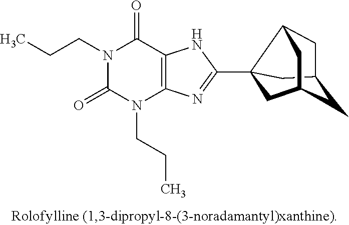

PSB-36 has the following chemical structure:

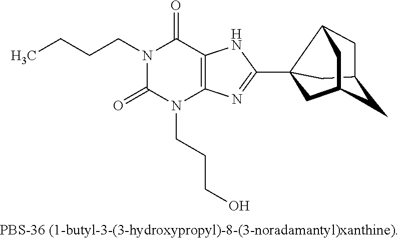

Naxifylline has the following chemical structure:

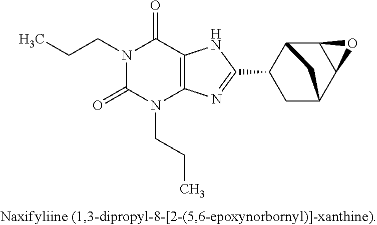 [SMILES: CCCn1c(=O)c2[nH]c([C@H]3C[C@@H]4C[C@H]3[C@H]3O[C@@H]43)nc2n(CCC)c1=O]

Toponafylline has the following chemical structure:

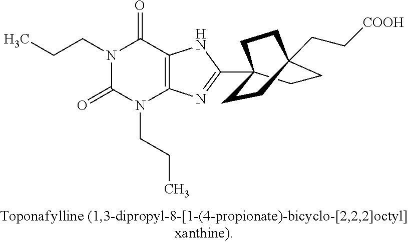

In further embodiments, the A₁-selective adenosine receptor antagonist that may be used in accordance with embodiments described herein is 3-[2-(4-aminophenyl)ethyl]-8-benzyl-7-[2-[ethyl(2-hydroxyethyl)amino]ethyl]-1-propylpurine-2,6-dione or 3-(2-(4-Aminophenyl)ethyl)-8-benzyl-7-(2-(ethyl(2-hydroxyethyl)amino)ethyl)-1-propylxanthine (also called “L-97-1”); L-97-1 has the following chemical structure:

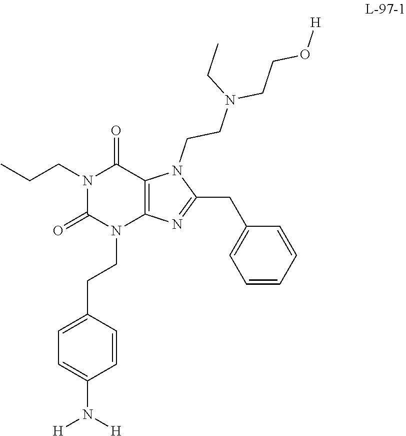 [SMILES: CCCn1c(=O)c2c(nc(Cc3ccccc3)n2CCN(CC)CCO)n(CCc2ccc(N)cc2)c1=O]

In some embodiments, the A1-selective adenosine receptor antagonists that may be used in accordance with embodiments described herein have a non-xanthine chemical structure, including but not limited to FK-453, which is a pyrazolopyridine derivative, SLV320, which is a 7-deazaadenine derivative (Muller and Jacobson, Biochim Biophys Acta. 2011 May; 1808(5): 1290-1308, which is hereby incorporated by reference in its entirety); their respective chemical structures are as follows:

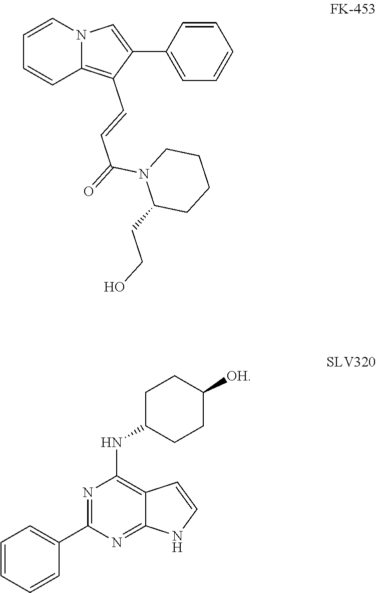

In another embodiment, A1-selective antagonist having a non-xanthine chemical structure that may be used in accordance with embodiments described herein is a 2-aminothiazole derivative, 2-benzoylamino-5-p-methylbenzoyl-4-phenylthiazole (Scheiff, A. B., et al., Bioorg Med Chem. 2010; 18:2195-2203, which is hereby incorporated by reference in its entirety), having the following chemical structure:

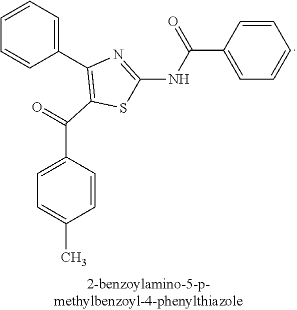 [SMILES: Cc1ccc(C(=O)c2sc(NC(=O)c3ccccc3)nc2-c2ccccc2)cc1]

### K+ Channel Inhibitors

The voltage-dependent potassium (K+) channel Kv1.3 is a transmembrane protein that is widely expressed throughout the body, however, it is highly expressed in both the nervous and immune systems. Kv1.3 has been regarded as a potential target for immunosuppression and as a therapeutic for autoimmune diseases dominated by the autoreactive effector memory T cell subset (T_{EM}). Psora-4 has been identified as a potent small-molecule Kv1.3 channel blocker, which preferentially binds to the C-type inactive state of Kv1.3, and also strongly blocks Kv1.5.

In some embodiments, the K+ channel inhibitor that may be used in accordance with embodiments described herein is a Kv1.3 potassium channel inhibitor (also called Kv1.3 blocker); the Kv channel blocker inhibits cAMP-stimulated neuritogenesis. In particular embodiments, the Kv1.3 potassium channel inhibitor is 5-(4-phenylbutoxy)psoralen (also called “Psora-4”), may be used in accordance with embodiments described herein; Psora-4 has the following chemical structure:

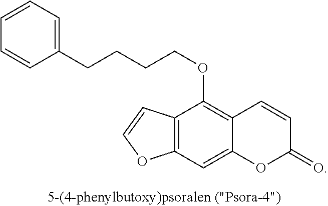 [SMILES: O=c1ccc2c(OCCCCc3ccccc3)c3ccoc3cc2o1]

Psora-4 analogs have been developed, as described by Vennenkamp et al. Mol. Pharmacol. 65:1364-1374, 2004, which is incorporated herein by reference in its entirety; Vennenkamp determined that compounds Psora-3, Psora-4, Psora-5 and Psora-9 were the most potent small-molecule Kv1.3 blockers. In some embodiments, the Kv1.3 potassium channel inhibitor is 5-(3-Phenylpropoxy)psoralen (also called “Psora-3”), which has the following chemical structure:

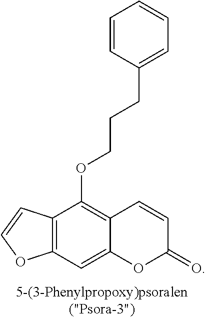 [SMILES: O=c1ccc2c(OCCCc3ccccc3)c3ccoc3cc2o1]

In another embodiment, the Kv1.3 potassium channel inhibitor is 5-(5-Phenylpentoxy)psoralen (also called “Psora-5”), which has the following chemical structure:

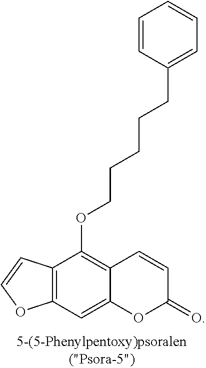 [SMILES: O=c1ccc2c(OCCCCCc3ccccc3)c3ccoc3cc2o1]

In a further embodiment, the Kv1.3 potassium channel inhibitor is 5-(4-Biphenylyl)-methoxypsoralen (also called “Psora-9”), which has the following chemical structure:

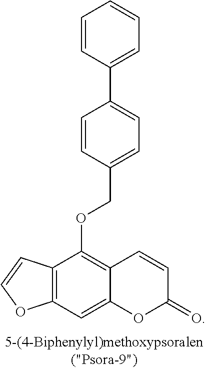 [SMILES: O=c1ccc2c(OCc3ccc(-c4ccccc4)cc3)c3ccoc3cc2o1]

In still further embodiments, the Kv1.3 potassium channel inhibitor that may be used in accordance with embodiments described herein is 5-(4-phenoxybutoxy)-psoralen (also called “phenoxyalkoxypsoralen-1” or “PAP-1”), which has the following chemical structure:

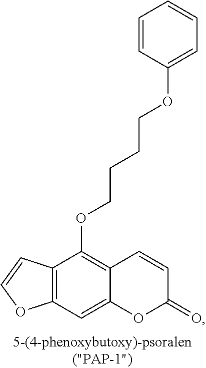 [SMILES: O=c1ccc2c(OCCCCOc3ccccc3)c3ccoc3cc2o1]

which has an increased selectivity for Kv1.3 over Kv1.5, as described by Schmitz, et al. Mol. Pharmacol. 68:1254-1270, 2005, which is incorporated herein by reference in its entirety.

### L-aromatic Amino Acid Decarboxylase Inhibitors

In some embodiments, the L-aromatic amino acid decarboxylase inhibitor that may be used in accordance with embodiments described herein is L-α-Methyl-3,4-dihydroxyphenylalanine, (also called “L-methyldopa”, “alpha-methyldopa” or “methyldopa”), which is an inhibitor of DOPA decarboxylase, an enzyme also known as “aromatic L-amino acid decarboxylase” (or “L-aromatic amino acid decarboxylase”), which converts DOPA into dopamine; methyldopa also is an alpha-2 adrenergic receptor agonist. L-methyldopa, which also is an alpha-2 adrenergic receptor agonist and decreases intracellular cAMP, has the following chemical structure:

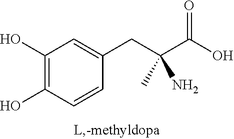

In certain embodiments, other L-aromatic amino acid decarboxylase inhibitors, including but not limited to carbidopa, benserazide (also called Serazide or Ro-4-4602), α-difluromethyldopa (DFMD), may be used in accordance with embodiments described herein; these DOPA decarboxylase inhibitors have the following chemical structures:

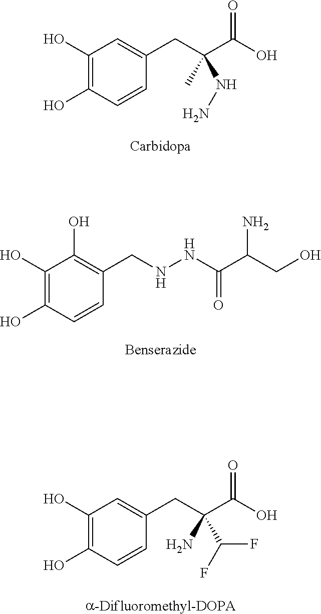 [SMILES: C[C@@](Cc1ccc(O)c(O)c1)(NN)C(=O)O ; NC(CO)C(=O)NNCc1ccc(O)c(O)c1O ; N[C@@](Cc1ccc(O)c(O)c1)(C(=O)O)C(F)F]

Analogs and derivatives of L-methyldopa also may be used in accordance with embodiments described herein, including but not limited to DL-α-methyl-α-hydrazino-3,4-dihydroxyphenyl-propionic acid (HMD), α-hydrazino-3,4-dihydroxyphenyl-propionic acid and α-hydrazino-3-hydroxyphenyl-propionic acid (Porter C. C., et al. Biochemical Pharmacology, 1962, Vol. 11, pp. 1067-1077, Pergamon Press Ltd., which is incorporated herein by reference in its entirety), having the following chemical structures:

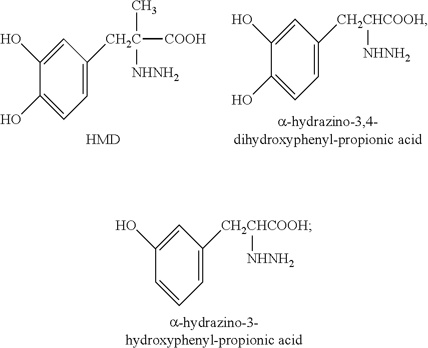

in addition, aromatic hydrazino acids, such as α-methyl-α-hydrazinophenyl-, α-methyl-α-hydrazino-(3-methoxy-4-hydroxyphenyl)-, and α-hydrazino-(4-methoxyphenyl)-propionic acids, described by Porter et al., which is incorporated herein by reference in its entirety, may be used as the DOPA decarboxylase inhibitor in accordance with embodiments described herein.

In various embodiments, to improve the absorption of orally administered L-methyldopa may be administered as a dipeptidyl derivative, such as, gly-L-α-methyldopa, pro-L-α-methyldopa, L-α-methyldopa-pro, phe-L-α-methyldopa, and L-α-methyldopa-L-phenylalanine (L-2-Methyl-3-(3,4-dihydroxyphenyl)alanyl-L-phenylalanine), as described by Hu, M., et al., Pharmaceutical Res., Vol. 6, No. 1, 1989, pp. 66-70, which is incorporated herein by reference in its entirety, or as dipeptides containing D-phenylglycine or D-p-hydroxyphenylglycine attached onto α-methyldopa to form prodrug, as described by Wang H-P., et al. J. Chinese Chem. Soc., 1995, which is incorporated herein by reference in its entirety.

### Imidazoline-1 Receptor Agonists

In various embodiments, the imidazoline agonist is clonidine hydrochloride (also called “clonidine”); clonidine stimulates alpha-2 adrenergic receptors and central imidazoline-1 (I₁) receptors; the hydrochloride salt of clonidine has the following chemical structure:

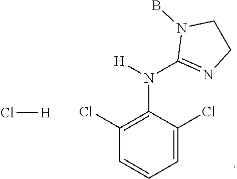

In alternate embodiments, the imidazoline agonist may be a clonidine analog, including but not limited to ICI-106,270, UK-14,304, piclonidine (LR-99,853), and the bridge analogs (ST-1913, ST-1966, ST-1967), as described by Sweet CS, Hypertension 1984 September-October; 6(5Pt 2):1151-6, which is incorporated herein by reference in its entirety.

In additional embodiments, the imidazoline agonist that may be used in accordance with embodiments described herein include, but are not limited to the following compounds:

naphazoline hydrochloride, which is an α-adrenoceptor agonist and an imidazoline receptor agonist having the following chemical structure:

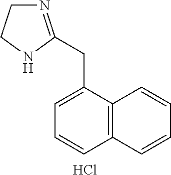 [SMILES: c1ccc2c(CC3=NCCN3)cccc2c1 ; Cl]

xylometazoline hydrochloride, which is an α-adrenoceptor agonist and imidazoline binding site ligand, having the following chemical structure:

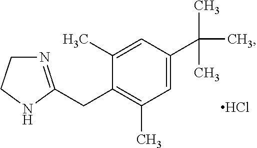 [SMILES: Cc1cc(C(C)(C)C)cc(C)c1CC1=NCCN1 ; Cl]

moxonidine hydrochloride, which is an α₂-adrenoceptor agonist and I₁ imidazoline binding site agonist that has shown selectivity for the high-affinity I₁ imidazoline binding site over the α₂-adrenoceptor, and has the following chemical structure:

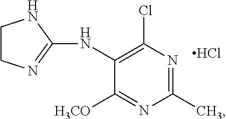 [SMILES: COc1nc(C)nc(Cl)c1NC1=NCCN1 ; Cl]

rilmenidine hemifumarate, which is an I₁-imidazoline binding site selective ligand and α₂-adrenoceptor agonist with greater I₁ receptor vs α₂-adrenoceptor selectivity than clonidine, having the following chemical structure:

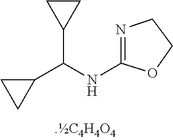 [SMILES: C1COC(NC(C2CC2)C2CC2)=N1]

In certain embodiments, 2-aminothiazoline derivatives that activate I1 imidazoline and alpha-2 adrenergic receptors have the general chemical structure:

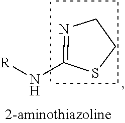

as described by Ferreira, R. B., et al., Eur. J. Pharmacol. 791, October 2016, 10.1016/j.ejphar.2016.10.009, which is incorporated herein by reference in its entirety. Ferreira, describes 2-aminothiazoline derivatives activating I₁-imidazoline and alpha-2 adrenergic receptors, which may be used in accordance with embodiments described herein. 2-aminothiazoline derivatives that may be used in accordance with embodiments described herein, include but are not limited to, N-substituted 2-aminothiazolines, such as diethyl and 2-ethyl-hexylamine derivatives, whose chemical structures, as shown in Ferreira, are as follows:

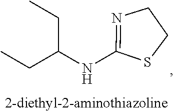

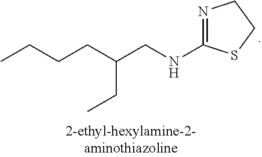

In some embodiments, the imidazoline-1 receptor agonist that may be used in accordance with embodiments described herein is a heterocyclic amine called harmane (also called 1-Methyl-9H-pyrido[3,4-b]indole, “2-methyl-b-carboline”, “harman” and “MS-1500866”), which is an imidazoline-1 (“I₁”) receptor agonist and also an alpha-2 adrenergic receptor agonist. Harmane has the following chemical structure:

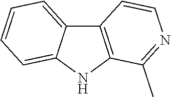

In additional embodiments, agmatine (the decarboxylated product of L-arginine), which is an agonist of alpha2-adrenergic receptors and imidazoine-1 (I₁) receptors, with a preferential affinity for human I₁ receptors and comparable affinity for alpha2A, alpha2B and alpha2C adrenoreceptors, may be used in accordance with embodiments described herein; agmatine has the following chemical structure:

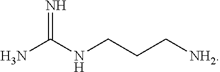

In alternate embodiments, imidazoline compounds that may be used in accordance with embodiments described herein activate I₁ imidazoline receptors and have little or no activity at the α2-adrenoreceptors; such I₁ imidazoline receptor agonists, include marsanidine and its analogs, 7-Me-marsanidine, 7-Cl-marsanidine, and 7-F-marsanidine and analogs or derivatives thereof.

In an embodiment, the imidazoline agonist is marsanidine, which has the following chemical structure:

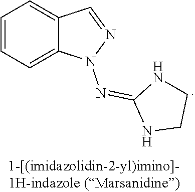 [SMILES: c1ccc2c(c1)cnn2N=C1NCCN1]

In another embodiment, the imidazoline agonist is 7-Me-marsanidine, which has the following chemical structure:

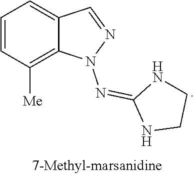 [SMILES: Cc1cccc2cnn(N=C3NCCN3)c12]

In another embodiment, the imidazoline agonist is 7-Cl-marsanidine, which has the following chemical structure:

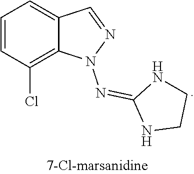 [SMILES: Clc1cccc2cnn(N=C3NCCN3)c12]

In another embodiment, the imidazoline agonist is 7-F-marsanidine, which has the following chemical structure:

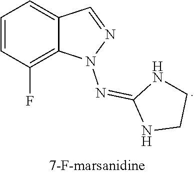 [SMILES: Fc1cccc2cnn(N=C3NCCN3)c12]

Of these marsanidine/marsanidine analog imidazoline compounds the 7-Me-marsanidine has the highest affinity for the I₁ imidazoline receptor with a low affinity for α2-adrenoreceptors, followed by 7-Cl-marsanidine, which has a lower affinity for the I₁ imidazoline receptor but a higher affinity for α2-adrenoreceptors, whereas 7-F-marsanidine has higher affinity for the I₁ imidazoline receptor than 7-Cl-marsanidine, but a comparable affinity for α2-adrenoreceptors; marsanidine has the highest affinity for α2-adrenoreceptors and the lowest affinity for the I₁ imidazoline receptor, and thus appears to be an α2-adrenoreceptor agonist rather than a mixed α2/I₁ agonist. (Boblewski et al., Acta Pol Pharm—Drug Research, Vol. 74, No. 2, pp. 579-586, 2017, which is incorporated herein by reference in its entirety).

### Serotonin Receptor Antagonists

In some embodiments, a serotonin receptor antagonist may be used may be used in accordance with embodiments described herein. In an embodiment, the serotonin antagonist is a semisynthetic ergot alkaloid that is a competitive alpha1-adrenergic receptor blocker and a partial alpha2-adrenergic receptor agonist. In various embodiments, the ergot alkaloid that is a serotonin (5-hydroxytryptamine or “5-HT”) receptor antagonist, is methysergide (also called “1-methyl-D-lysergic acid butanolamide”, “UML-491”, and “methysergide maleate”, a salt thereof), which is a serotonin 5-HT_{2c} receptor antagonist having the following chemical structure:

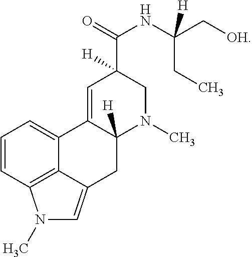

In another embodiment, the serotonin receptor antagonist is amesergide (also called “N-Cyclohexyl-11-isopropyllysergamide” and “LY-237733”), which is a selective antagonist of serotonin 5-HT_{2A}, 5-HT_{2B}, and 5-HT_{2C} receptors and a potent antagonist of the α₂-adrenergic receptor; amesergide is related to methysergide and has the following chemical structure:

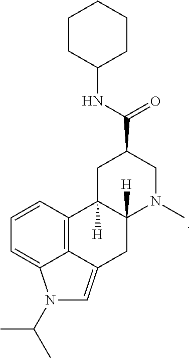

In an embodiment, the semisynthetic ergot alkaloid that is a serotonin receptor antagonist is methylergometrine (also called “methylergonovine”, “methylergobasin”, “D-lysergic acid 1-butanolamide” and its salt methylergonovine maleate (Methergine®)), an active metabolite of methysergide, that is a partial agonist/antagonist of serotonergic, dopaminergic and alpha-adrenergic receptors and has the following chemical structure:

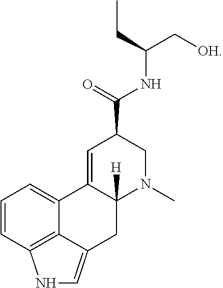

### Cyclic Nucleotide Phosphodiesterase Inhibitors

In an embodiment, cyclic nucleotide phosphodiesterase (“PDE”) inhibitors may be used may be used in accordance with embodiments described herein. PDE3 is a cGMP-inhibited phosphodiesterase. In a particular embodiment, the PDE inhibitor is the selective phosphodiesterase 3 (PDE3) inhibitor cilostazol (also called “6-[4-(1-cyclohexyl-1H-tetrazol-5-yl)-butoxy]-3,4-dihydro-2(1H)-quinolinone”, “MS-1505230”, and “PLETAAL®”), which increases cAMP by suppressing cAMP degradation, resulting in an increase in the active form of protein kinase A (PKA) and is related to inhibition of platelet aggregation; cilostazol has the following chemical formula:

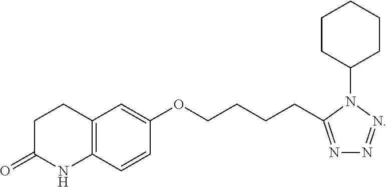 [SMILES: O=C1CCc2cc(OCCCCc3nnnn3C3CCCCC3)ccc2N1]

In some embodiments, cilostazol analogs and pharmaceutically acceptable salts thereof, as described in U.S. Pat. No. 8,349,817, which is incorporated in its entirety, may be used as a PDE3 inhibitor in accordance with embodiments described herein, including but not limited to the following compounds:

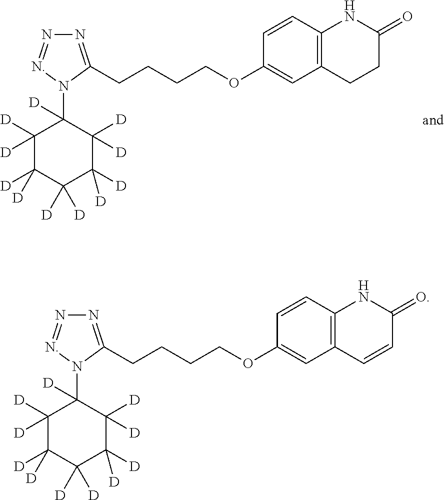

wherein D is deuterium.

In another embodiment, the PDE3 inhibitor that may be used in accordance with embodiments described herein is milrinone (6-methyl-2-oxo-5-pyridin-4-yl-1H-pyridine-3-carbonitrile), amrinone (3-amino-5-pyridin-4-yl-1H-pyridin-2-one, also called “inamrinone”), pelrinone (2-methyl-4-oxo-6-(pyridin-3-ylmethylamino)-1H-pyrimidine-5-carbonitrile), enoximone (4-methyl-5-(4-methylsulfanylbenzoyl)-1,3-dihydroimidazol-2-one), pimobendan (4,5-dihydro-6-(2-(4-methoxyphenyl)-1H-benzimidazole-5-yl)-5-methyl-3(2H)-pyridazinone) or meribendan (4,5-Dihydro-5-methyl-6-(2-pyrazol-3-yl-5-benzimidazolyl)-3(2H)-pyridazinone), which have the following chemical structures, respectively:

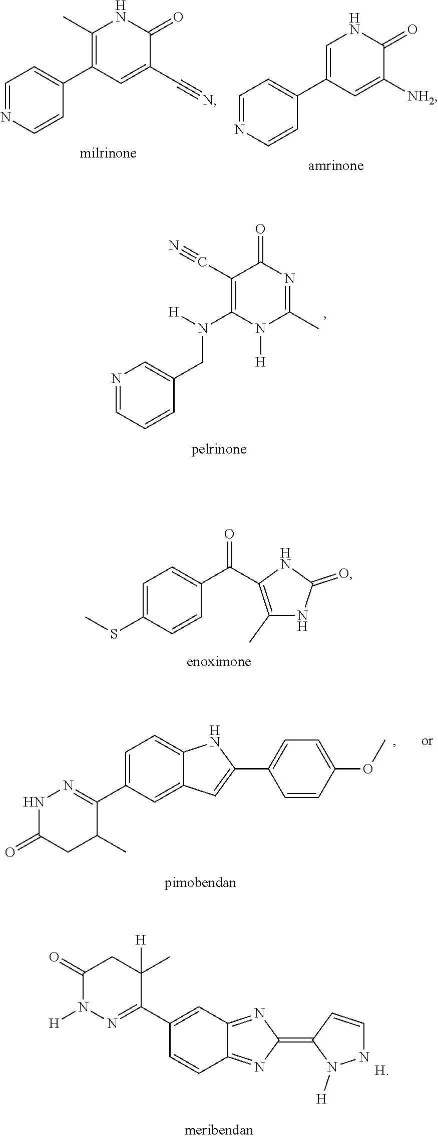

In some embodiments, the PDE3 inhibitor is cilostamide (6-[3-(N-Cyclohexyl-N-methylcarbamoyl)propoxy]quinolin-2[1H]-one), a selective inhibitor of PDE3 which has the following chemical structure:

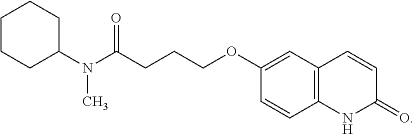 [SMILES: CN(C(=O)CCCOc1ccc2[nH]c(=O)ccc2c1)C1CCCCC1]

In additional embodiments, the PDE3 inhibitor is the extremely potent PDE3 inhibitor trequinsin or its salt trequinsin hydrochloride (2,3,6,7-tetrahydro-9,10-dimethoxy-3-methyl-2-[(2,4,6-trimethylphenyl)imino]-4H-pyrimido[6,1-a]isoquinolin-4-one hydrochloride), which has the following chemical structure:

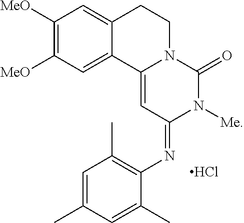 [SMILES: COc1cc2c(cc1OC)-c1c/c(=N\c3c(C)cc(C)cc3C)n(C)c(=O)n1CC2 ; Cl]

In another embodiment, the PDE3 inhibitor is a cyclooctylurea derivative (and a cilostamide analog) named OPC-33540 (6-[3-[3-cyclooctyl-3-[(1R*,2R*)-2 hydroxycycloexyl]ureido]-propoxy]-2(1H)-quinolinone), which has been found to be more potently and selectively than the classical PDE3 inhibitors cilostamide, cilostazol, milrinone, and amrinone, as described by Sudo et al. Biochem Pharmacol. 2000 Feb. 15; 59(4):347-56, which is incorporated in its entirety. OPC-33540 has the following chemical structure:

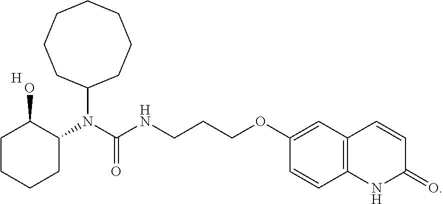

In certain embodiments, the implant comprises a metal titanium, a titanium alloy, chrome or steel. In another embodiment, the implant comprises a smooth surface and/or a complex surface. In various embodiments, the Npas2 modulating compound is administered into the bone marrow concurrently with implantation of the implant at an implant location. In a further embodiment, the Npas2 modulating compound is administered into the bone marrow before implantation of the implant. In another embodiment, the Npas2 modulating compound is administered into the bone marrow after implantation of the implant. In an embodiment, the Npas2 modulating compound is coated onto the implant prior to implantation thereof. In various embodiments, the Npas2 modulating compound upregulates Npas2. In an embodiment, Npas2 upregulation decreases intracellular cAMP. In various embodiments, Npas2 upregulation stimulates α2 adrenergic receptor expression. In some embodiments, the α2 adrenergic receptor is an α2A-, α2B- and/or an α2C-adrenergic receptor.

In embodiments, the Npas2 modulating compound is an adenosine receptor antagonist, the adenosine receptor antagonist having selectivity for adenosine receptor A1 over adenosine receptor A2. In an embodiment, the adenosine receptor antagonist is 8-(p-sulfophenyl) theophylline.

In some embodiments, the adenosine A1 receptor antagonist is selected from the group consisting of 1,3-dipropyl-8-phenylxanthine, 8-(2-amino-4-chlorophenyl)-1,3-dipropylxanthine, 1-isoamyl-3-isobutylxanthine, (R)-3,7-dihydro-8-(1-methyl-2-phenylethyl)-1,3-dipropyl-1H-purine-2,6-dione, (R)-3,7-dihydro-8-(1-phenylpropyl)-1,3-dipropyl-1H-purine-2,6-dione, 1,3-dipropyl-8-cyclopentylxanythine (DPCPX), 8-Cyclopentyl-1,3-dimethylxanthine (CPX), 1,3-dipropyl-8-(3-noradamantyl)xanthine (rolofylline), 1-butyl-3-(3-hydroxypropyl)-8-(3-noradamantyl)xanthine (PSB-36), 1,3-dipropyl-8-[2-(5,6-epoxynorbonyl)]-xanthine (naxifylline), dicyclopropylmethyl (MPDX), 1,3-dipropyl-8-[1-(4-propionate)-bicyclo-[2,2,2]octyl]xanthine (toponafylline), 3-(2-(4-Aminophenyl)ethyl)-8-benzyl-7-(2-(ethyl(2-hydroxyethyl)amino)ethyl)-1-propylxanthine (L-97-1) and analogs or salts thereof.

In certain embodiments, the adenosine A1 receptor antagonist is a non-xanthine compound selected from the group consisting of 2-aminothiazole derivatives.

In various embodiments, the Npas2 modulating compound is a Kv1.3 potassium channel inhibitor. In an embodiment, the Kv1.3 potassium channel inhibitor is 5-(4-phenylbutoxy)psoralen (also called “Psora-4”). In another embodiment, the Kv1.3 potassium channel inhibitor is selected from the group consisting of 5-(3-Phenylpropoxy)psoralen (also called “Psora-3”), 5-(5-Phenylpentoxy)psoralen (also called “Psora-5”), 5-(4-Biphenylyl)-methoxypsoralen (also called “Psora-9”) and 5-(4-phenoxybutoxy)-psoralen (also called “PAP-1”).

In some embodiments, the Npas2 modulating compound is an L-aromatic amino acid decarboxylase inhibitor. In an embodiment, the L-aromatic amino acid decarboxylase inhibitor further is an α2 adrenergic receptor agonist, wherein the compound is L-methyldopa or an analog or derivative thereof. In additional embodiments, the L-aromatic amino acid decarboxylase inhibitor is selected from the group consisting of carbidopa, benserazide α-difluromethyldopa and analogs thereof.

In further embodiments, the Npas2 modulating compound is an imidazoline-1 receptor agonist. In another embodiment, the imidazoline-1 receptor agonist is harmane (MS-1500866). In some embodiments, the imidazoline-1 receptor agonist is selected from the group consisting of clonidine hydrochloride or clonidine analog, naphazoline hydrochloride, naphazoline hydrochloride, xylometazoline hydrochloride, moxonidine hydrochloride, rilmenidine hemifumarate, a 2-aminothiazoline derivative, and an analog or derivative thereof. In additional embodiments, the 2-aminothiazoline derivative is selected from the group consisting of 2-diethyl-2-aminothiazoline, 2-ethyl-hexylamine-2-aminothiazoline and an analog or derivative thereof. In some embodiments, the imidazoline-1 receptor agonist is selected from the group consisting of marsanidine, 7-methyl-marsanidine, 7-Cl-marsanidine, 7-F-marsanidine and an analog or derivative thereof. In some embodiments, the smooth surface implant further comprises a complex surface. In embodiments, the complex surface is prepared by sandblasting with large-grit and acid-etching (SLA).

In some embodiments, the Npas2 upregulation occurs in human bone marrow stromal cells (BMSC) exposed to a surface of the implant, wherein the surface is a smooth surface and/or a complex surface. In another embodiment, the Npas2 upregulation in BMSC facilitates bonding of bone and implant surface at an interface tissue between the bone and the implant. In various embodiments, the Npas2 upregulation in BMSC stimulates synthesis of dense collagen fibers on the interface tissue, wherein the collagen structure is crisscrossed. In some embodiments, the Npas2 upregulation in BMSC further stimulates synthesis of dense collagen fibers on the implant surface, wherein the collagen structure is crisscrossed. This collagen structure is synthesized in the interface tissue.

In various embodiments, the implant is a dental implant or an orthopedic implant. In some embodiments, the expression of NPAS2 is increased by administering to the subject an adenoviral vector, the adenoviral vector comprising a nucleic acid encoding a human NPAS2 polypeptide. In certain embodiments, the adenoviral vector is administered to the subject at an implant location. In some embodiments, the adenoviral vector is administered into the bone marrow concurrently with implantation of the implant at the implant location, before implantation of the implant and/or after implantation of the implant.

In another aspect, the invention provides a method for accelerating osseointegration of an implant into bone marrow of a subject, the method comprising administering to the subject a pharmaceutical composition comprising an NPAS2 polypeptide or an Npas2 modulating compound, wherein the NPAS2 polypeptide or the Npas2 modulating compound increases expression of peripheral clock neuronal PAS domain protein 2 (NPAS2) in the bone marrow. In some embodiments, the implant comprises titanium, a titanium alloy, chrome or steel. In certain embodiments, the implant comprises the implant comprises a smooth surface and/or a complex surface.

In various embodiments, expression of NPAS2 is increased by administration of a Npas2 modulating compound to the subject. In another embodiment, the Npas2 modulating compound is administered into the bone marrow concurrently with implantation of the implant at an implant location. In a further embodiment, the Npas2 modulating compound is administered into the bone marrow before implantation of the implant. In another embodiment, the Npas2 modulating compound is administered into the bone marrow after implantation of the implant. In an embodiment, the Npas2 modulating compound is coated onto the implant prior to implantation thereof. In various embodiments, the Npas2 modulating compound upregulates Npas2. In an embodiment, Npas2 upregulation decreases intracellular cAMP. In various embodiments, Npas2 upregulation stimulates α2 adrenergic receptor expression. In some embodiments, the α2 adrenergic receptor is an α2A-, α2B- and/or an α2C-adrenergic receptor.

In some embodiments, the Npas2 modulating compound is an adenosine receptor antagonist, the adenosine receptor antagonist having selectivity for adenosine receptor A1 over adenosine receptor A2. In another embodiment, the adenosine receptor antagonist is 8-(p-sulfophenyl) theophylline.

In additional embodiments, the adenosine A1 receptor antagonist is selected from the group consisting of 1,3-dipropyl-8-phenylxanthine, 8-(2-amino-4-chlorophenyl)-1,3-dipropylxanthine, 1-isoamyl-3-isobutylxanthine, (R)-3,7-dihydro-8-(1-methyl-2-phenylethyl)-1,3-dipropyl-1H-purine-2,6-dione, (R)-3,7-dihydro-8-(1-phenylpropyl)-1,3-dipropyl-1H-purine-2,6-dione, 1,3-dipropyl-8-cyclopentylxanythine (DPCPX), 8-Cyclopentyl-1,3-dimethylxanthine (CPX), 1,3-dipropyl-8-(3-noradamantyl)xanthine (rolofylline), 1-butyl-3-(3-hydroxypropyl)-8-(3-noradamantyl)xanthine (PSB-36), 1,3-dipropyl-8-[2-(5,6-epoxynorbonyl)]-xanthine (naxifylline), dicyclopropylmethyl (MPDX), 1,3-dipropyl-8-[1-(4-propionate)-bicyclo-[2,2,2]octyl]xanthine (toponafylline), 3-(2-(4-Aminophenyl)ethyl)-8-benzyl-7-(2-(ethyl(2-hydroxyethyl)amino)ethyl)-1-propylxanthine (L-97-1) and analogs or salts thereof.

In some embodiments, the adenosine A1 receptor antagonist is a non-xanthine compound selected from the group consisting of 2-aminothiazole derivatives.

In various embodiments, the Npas2 modulating compound is a Kv1.3 potassium channel inhibitor. In an embodiment, the Kv1.3 potassium channel inhibitor is 5-(4-phenylbutoxy)psoralen (also called “Psora-4”). In another embodiment, the Kv1.3 potassium channel inhibitor is selected from the group consisting of 5-(3-Phenylpropoxy)psoralen (also called “Psora-3”), 5-(5-Phenylpentoxy)psoralen (also called “Psora-5”), 5-(4-Biphenylyl)-methoxypsoralen (also called “Psora-9”) and 5-(4-phenoxybutoxy)-psoralen (also called “PAP-1”).

In some embodiments, the Npas2 modulating compound is an L-aromatic amino acid decarboxylase inhibitor. In an embodiment, the L-aromatic amino acid decarboxylase inhibitor further is an α2 adrenergic receptor agonist, wherein the compound is L-methyldopa or an analog or derivative thereof. In various embodiments, the L-aromatic amino acid decarboxylase inhibitor is selected from the group consisting of carbidopa, benserazide α-difluromethyldopa and analogs thereof.

In further embodiments, the Npas2 modulating compound is an imidazoline-1 receptor agonist. In another embodiment, the imidazoline-1 receptor agonist is harmane (MS-1500866). In some embodiments, the imidazoline-1 receptor agonist is selected from the group consisting of clonidine hydrochloride or clonidine analog, naphazoline hydrochloride, naphazoline hydrochloride, xylometazoline hydrochloride, moxonidine hydrochloride, rilmenidine hemifumarate, a 2-aminothiazoline derivative, and an analog or derivative thereof. In additional embodiments, the 2-aminothiazoline derivative is selected from the group consisting of 2-diethyl-2-aminothiazoline, 2-ethyl-hexylamine-2-aminothiazoline and an analog or derivative thereof. In some embodiments, the imidazoline-1 receptor agonist is selected from the group consisting of marsanidine, 7-methyl-marsanidine, 7-Cl-marsanidine, 7-F-marsanidine and an analog or derivative thereof. In some embodiments, the implant comprises a smooth surface and/or a complex surface. In embodiments, the complex surface is prepared by sandblasting with large-grit and acid-etching (SLA).

In additional embodiments, the expression of NPAS2 further is increased by administering to the subject an adenoviral vector, the adenoviral vector comprising a nucleic acid encoding a human NPAS2 polypeptide. In some embodiments, the adenoviral vector is administered into the bone marrow concurrently with implantation of the smooth surface implant at the implant location. In additional embodiments, the adenoviral vector is administered into the bone marrow before implantation of the smooth surface implant. In further embodiments, the adenoviral vector is after implantation of the smooth surface implant.

In another aspect, the invention provides a method for re-establishing an implant-bone integration in a subject, the method comprising increasing expression of peripheral clock neuronal PAS domain protein 2 (NPAS2) in the bone marrow.

In a further aspect, the invention provides a method for improving osseointegration of a titanium implant into bone marrow of a subject, the method comprising administering to the subject a pharmaceutical composition comprising a NPAS2 polypeptide or a Npas2 modulating compound, wherein the Npas2 modulating compound increases expression of peripheral clock neuronal PAS domain protein 2 (NPAS2) in the bone marrow.

In another aspect, the invention provides a method for improving or accelerating bone repair and wound healing, the method comprising administering to the subject a pharmaceutical composition comprising a NPAS2 polypeptide or a Npas2 modulating compound, wherein the Npas2 modulating compound increases expression of peripheral clock neuronal PAS domain protein 2 (NPAS2) in bone marrow and wound tissue.

In an embodiment, the pharmaceutical composition comprising a NPAS2 polypeptide or a Npas2 modulating compound is administered directly to a wound site or a bone fracture.

In some embodiments, the wound comprises epithelial tissue, muscle tissue, connective tissue or nervous tissue, e.g., peripheral nervous system (PNS) tissue or central nervous tissue (CNS). In various embodiments, the epithelial tissue comprises cutaneous (skin) tissue or a lining of gastrointestinal tract organs and other hollow organs and certain glands. In embodiments, muscle tissue comprises smooth muscle tissue, cardiac muscle tissue or skeletal muscle tissue. Connective tissue may be connective tissue proper (loose connective tissue and dense connective tissue) or special connective tissue (reticular connective tissue, adipose tissue, cartilage, bone, and blood). In some embodiments, the connective tissue comprises fibers (elastic and collagenous fibers), ground substance and cells; connective tissue cells comprises fibroblasts, adipocytes, macrophages, mast cells and leucocytes. In some embodiments, the pharmaceutical composition comprising a NPAS2 polypeptide or a Npas2 modulating compound is administered immediately after injury, i.e., from within seconds up to hours (e.g., from 1 to 7 hours post-injury), during inflammation of the wound (from within hours up to days, from 1 to 3 days post-injury), or during the repair process (from days to weeks post-injury).

In some embodiments of these provided methods, the expression of NPAS2 is increased by administration of a Npas2 modulating compound to the subject. In another embodiment, the Npas2 modulating compound is administered into the bone marrow concurrently with implantation of the titanium implant at an implant location. In a further embodiment, the Npas2 modulating compound is administered into the bone marrow before implantation of the titanium implant. In another embodiment, the Npas2 modulating compound is administered into the bone marrow after implantation of the titanium implant. In an embodiment, the Npas2 modulating compound is coated onto the titanium implant prior to implantation thereof. In various embodiments, the Npas2 modulating compound upregulates Npas2. In an embodiment, Npas2 upregulation decreases intracellular cAMP. In various embodiments, Npas2 upregulation stimulates α2 adrenergic receptor expression. In some embodiments, the α2 adrenergic receptor is an α2A-, α2B- and/or an α2C-adrenergic receptor.

In certain embodiments, the Npas2 modulating compound is an adenosine receptor antagonist, the adenosine receptor antagonist having selectivity for adenosine receptor A1 over adenosine receptor A2. In another embodiment, the adenosine receptor antagonist is 8-(p-sulfophenyl) theophylline.

In additional embodiments, the adenosine A1 receptor antagonist is selected from the group consisting of 1,3-dipropyl-8-phenylxanthine, 8-(2-amino-4-chlorophenyl)-1,3-dipropylxanthine, 1-isoamyl-3-isobutylxanthine, (R)-3,7-dihydro-8-(1-methyl-2-phenylethyl)-1,3-dipropyl-1H-purine-2,6-dione, (R)-3,7-dihydro-8-(1-phenylpropyl)-1,3-dipropyl-1H-purine-2,6-dione, 1,3-dipropyl-8-cyclopentylxanythine (DPCPX), 8-Cyclopentyl-1,3-dimethylxanthine (CPX), 1,3-dipropyl-8-(3-noradamantyl)xanthine (rolofylline), 1-butyl-3-(3-hydroxypropyl)-8-(3-noradamantyl)xanthine (PSB-36), 1,3-dipropyl-8-[2-(5,6-epoxynorbonyl)]-xanthine (naxifylline), dicyclopropylmethyl (MPDX), 1,3-dipropyl-8-[1-(4-propionate)-bicyclo-[2,2,2]octyl]xanthine (toponafylline), 3-(2-(4-Aminophenyl)ethyl)-8-benzyl-7-(2-(ethyl(2-hydroxyethyl)amino)ethyl)-1-propylxanthine (L-97-1) and analogs or salts thereof.

In some embodiments, the adenosine A1 receptor antagonist is a non-xanthine compound selected from the group consisting of 2-aminothiazole derivatives.

In various embodiments, the Npas2 modulating compound is a Kv1.3 potassium channel inhibitor. In an embodiment, the Kv1.3 potassium channel inhibitor is 5-(4-phenylbutoxy)psoralen (also called “Psora-4”). In another embodiment, the Kv1.3 potassium channel inhibitor is selected from the group consisting of 5-(3-Phenylpropoxy)psoralen (also called “Psora-3”), 5-(5-Phenylpentoxy)psoralen (also called “Psora-5”), 5-(4-Biphenylyl)-methoxypsoralen (also called “Psora-9”) and 5-(4-phenoxybutoxy)-psoralen (also called “PAP-1”).

In further embodiments, the Npas2 modulating compound is an L-aromatic amino acid decarboxylase inhibitor. In an embodiment, the L-aromatic amino acid decarboxylase inhibitor further is an α2 adrenergic receptor agonist, wherein the compound is L-methyldopa or an analog or derivative thereof. In various embodiments, the L-aromatic amino acid decarboxylase inhibitor is selected from the group consisting of carbidopa, benserazide α-difluromethyldopa and analogs thereof.

In some embodiments, the Npas2 modulating compound is an imidazoline-1 receptor agonist. In another embodiment, the imidazoline-1 receptor agonist is harmane (MS-1500866). In some embodiments, the imidazoline-1 receptor agonist is selected from the group consisting of clonidine hydrochloride or clonidine analog, naphazoline hydrochloride, naphazoline hydrochloride, xylometazoline hydrochloride, moxonidine hydrochloride, rilmenidine hemifumarate, a 2-aminothiazoline derivative, and an analog or derivative thereof. In additional embodiments, the 2-aminothiazoline derivative is selected from the group consisting of 2-diethyl-2-aminothiazoline, 2-ethyl-hexylamine-2-aminothiazoline and an analog or derivative thereof. In some embodiments, the imidazoline-1 receptor agonist is selected from the group consisting of marsanidine, 7-methyl-marsanidine, 7-Cl-marsanidine, 7-F-marsanidine and an analog or derivative thereof. In some embodiments, the implant comprises a smooth surface and/or a complex surface. In embodiments, the complex surface is prepared by sandblasting with large-grit and acid-etching (SLA).

In additional embodiments of the provided methods, the expression of NPAS2 is increased by administering to the subject an adenoviral vector, the adenoviral vector comprising a nucleic acid encoding a human NPAS2 polypeptide. In some embodiments, the adenoviral vector is administered to the subject at an implant location. In various embodiments, the adenoviral vector is administered into the bone marrow concurrently with implantation of the titanium implant at the implant location. In embodiments, the adenoviral vector is administered into the bone marrow before implantation of the titanium implant. In some embodiments, the adenoviral vector is administered into the bone marrow after implantation of the titanium implant.

In some embodiments of the provided methods, the implant may comprise a smooth surface, a complex surface or a combination thereof. In various embodiments, the complex surface is prepared by sandblasting with large-grit and acid-etching (SLA). In particular embodiments, Npas2 upregulation occurs in human bone marrow stromal cells (BMSC) exposed to the implant surface. In an embodiment, the Npas2 upregulation in BMSC facilitates bonding of bone and the implant at an interface tissue between the bone and the implant. In another embodiment, the Npas2 upregulation in BMSC stimulates synthesis of dense collagen fibers on the interface tissue, wherein the collagen structure is crisscrossed. In further embodiments, the Npas2 upregulation in BMSC further stimulates synthesis of dense collagen fibers on the implant, wherein the collagen structure is crisscrossed.

### Co-Administered Drugs for Promoting/Increasing Osseointegration

The osseointegration of an implant, i.e., successful bone-to-implant integration, may be enhanced by depositing bioactive drugs onto the implant surface or co-administering a bioactive drug to a subject receiving an implant. In some embodiments, bioactive drugs may be used in accordance with embodiments described herein, i.e., are deposited or coated on the implant surface or co-administered to the subject. In certain embodiments, the bioactive drugs comprises calcium phosphate, which is similar to the natural bone mineral, extracellular matrix (ECM) proteins, such as collagen, including collagen type-1, and elastin, enzymes, and growth factors, such as bone morphogenetic proteins (BMPs) and/or transforming growth factor-β1 (TGF-β1), and bone morphogenetic proteins (BMPs), including recombinant human BMPs, such as rhBMP-2 and rhBMP-7 (Alghamdi, H. S., J. Funct. Biomater. 2018, 9,7; doi:10.3390/jfb9010007, which is hereby incorporated by reference in its entirety.).

In additional embodiments, pharmacological drugs may be used in accordance with embodiments described herein, include but are not limited to, antiresorptive drugs, such as biophosphonates, and anabolic drugs, such as, strontium ranelate and statins, are deposited onto the surface of the implant or are co-administered to the subject before, during or after implantation of the implant. (Alghamdi, H. S., J. Funct. Biomater. 2018, 9,7; Maimun, L., et al., Bone. 2010 May; 46(5): 1436-41, which are hereby incorporated by reference in their entirety). In some embodiments, an implant coating that may be used in accordance with embodiments described herein is a biophosphonate (BP)-releasing coating, wherein the pharmacological drug is a sustained release formulation comprising a BP (such as (i) alendronate in combination with HA, collagen 1, chondroitin sulfate and calcium phosphate; (ii) pamindronate and (iii) ibandronate, together or separately in combination with fibrinogen; or (iv) zoledronate with fibrinogen and calcium phosphate (Najeeb, S., et al., which is incorporated herein by reference in its entirety).

In further embodiments, an implant may be coated with one or more layer of calcium phosphates primarily comprising hydroxyapatite (HA), a mineral form of calcium apatite, which may be coated onto the implant by plasma-spraying coating method (Le Guehennec, L., et al., Dental Materials 23 (2007) 844-854, which is incorporated herein by reference in its entirety). In some embodiments, the HA coating may be applied onto the by a direct chemical method without need for subsequent heat treatment (Lukaszewska-Kuska, M., et al., Adv Clin Med. 2018; 27(8):1055-59, which is incorporated herein by reference in its entirety).

In some embodiments, the implant may be coated with the active agent, zoledronic acid, a potent bisphosphonate having a high affinity to mineralized bone. Coating a titanium implant with zoledronic acid was found to significantly increase bone-implant contact (BIC), peri-implant bone area (BA) surrounding the implant, bone volume/tissue volume, and bone-mineral density (BMD). (Stradlinger et al., European Cells and Materials Vol. 25 2013, pp. 326-40, which is incorporated herein by reference in its entirety).

In various embodiments, implant coatings that may be used in accordance with embodiments described herein are zirconium oxide coatings (“ZOC”) (prepared as a colloidal suspension to coat implant surfaces), which have been shown to have specific biologic effects, such as, more evident bone growth around the ZOC-coated implants and more mature bone present in the peri-implant ZOC surface than in the respective controls (Sollazzo, V., et al., Dental Materials, Vol. 24(3), March 2008, pp. 357-361., which is incorporated herein by reference in its entirety).

In some embodiments, a carbon film, e.g., with a chemical composition of Ti0.5O0.3C0.2, may be coated onto an implant; additional implant coatings that may be used in accordance with embodiments described herein, include but are not limited to bisphosphonates, bone stimulating factors (including BMPs, e.g., a polylactide/glycolide (PLGA) carrier comprising rhBMP-2; a platelet-derived growth factors (PDGFs) and insulinlike growth factors (IGFs) or a combination of PDGF-B and IGFs); bioactive glass and bioactive ceramics; bioactive implant coatings comprising fluoride; and titanium/titanium nitride coatings (Xuereb, M., et al., Intl J Prosthodontics, Vol 28, No. 1, 2015, which is incorporated herein by reference in its entirety).

In further embodiments, inhibitors of the epigenetic enzyme ‘Enhancer of Zeste homolog 2’ (“EZH2”), which stimulate new bone formation of the osteogenic pathway in mesenchymal stem cells may be used in accordance with embodiments described herein to coat an implant surface; EZH2 inhibitors include but are not limited to GSK126, a specific EZH2 inhibitor, which also enhances BMP-2-induced osteogenic differentiation, (Dudakovic et al. (2016) J Biol Chem 291(47):24594-24606, which is incorporated herein by reference in its entirety). In still further embodiments, small molecule drug compounds that induce osteogenesis and promote implant osseointegration, currently in development by Numerate, Inc. and the Mayo Clinic, may be used as implant coatings in accordance with embodiments described herein (National Institutes of Health Awards Grant to Numerate to Develop Compounds that Enhance Bone Integration of Orthopedic Devices, BioSpace, Business Wire, Nov. 15, 2017)

In certain embodiments, the implant surface comprises TiO₂ nanotube arrays, which comprise nanotubes comprising bioactive drugs to improve osseointegration, i.e., loaded in the nanotube arrays. In further embodiments, the titanium implant comprises TiO₂ nanotube arrays on the surface of the implant, wherein the TiO₂ nanotube arrays comprise rhBMP-2 that is eluted from the TiO₂ nanotube arrays (Lee, J-K., et al., Intl J Nanomedicine February 2015, Vol. 2015:10(1), pp. 1145-54, which is incorporated herein by reference in its entirety). In another embodiment, the TiO₂ nanotubes comprise propolis, a natural antibacterial and anti-inflammatory (Somsanith, N., et al., Materials (Basel) 2018, 11(1), 61, which is incorporated herein by reference in its entirety).

In further embodiments, fabrication of hierarchical microtopographic/nanotopographic coatings on nanograined titanium implants, employing the method of molecular layering of atomic layer deposition (ML-ALD), which has been found to improve osseointegration properties of titanium implants, may be used in accordance with embodiments described herein (Zemtsova, E. G., et al., Intl J Nanomedicine 2018: 13 2175-2188, which is incorporated herein by reference in its entirety).

In additional embodiments, a peptide coating may be used in accordance with embodiments described herein, i.e., may be applied onto an implant, e.g., titanium implant; in particular, a bifunctional peptide, composed of a β-strand decorated by two pSer residues that adsorb strongly on the oxide surface layer of titanium (TiO₂), followed by a Glu-rich ‘tail’ that induces calcified mineralization (Povimonsky, A. G. and H. Rapaport, J. Mater. Chem. B, 2017, 5, 2096-2105, which is incorporated herein by reference in its entirety). In further embodiments, a peptide coating that may be coated on an implant (e.g., a titanium implant) to improve osseointegration, wherein the peptide coating comprises a combination of two mussel-inspired bioactive peptides with cell adhesive or osteogenic sequences, respectively, as described by Zhao, H., et al., ACS Biomater. Sci. Eng., 2018, 4 (7), pp 2505-2515, which is incorporated herein by reference in its entirety).

In some embodiments, engineered protein coatings may be used in accordance with embodiments described herein, including but not limited to engineered elastin-like protein (ELP), which includes an extended RGD sequence, which was found to improve osseointegration of titanium-based implants (dental and orthopedic) (Raphel, J., et al., Biomaterials, March 2016, pp. 269-282, which is incorporated herein by reference in its entirety).

In certain embodiments, a calcium carbonate coating may be used in accordance with embodiments described herein on sandblasted and acid-etched titanium implants, which have been found to improve and accelerate early ingrowth of bone and osseointegration (Liu, Y., et al., Intl J Oral Science (2017) 9, 133-138, which is incorporated herein by reference in its entirety).

In another embodiment of the osseointegration improvement methods, a titanium implant surface may be treated (cleaned) with ozonated water; such treatment has been found to decrease osseointegration time; in embodiments, the implant surface may be cleaned with ozonated water before implantation (Yoshida, G., et al., J Hard Tissue Biology Vol. 2592):149-156, 2016, which is incorporated herein by reference in its entirety).

In some embodiments, methods of improving or accelerating osseointegration of an implant (e.g., titanium implant) and/or accelerating bone repair, may be enhanced by further methods of administration of therapeutically effective amounts of systemically delivered drugs, including but not limited to anabolic bone-acting agents, including parathyroid hormone (PTH) peptides, simvastatin, prostaglandin EP4 receptor antagonist, vitamin D and strontium ranelate; anti-catabolic bone-acting agents, including compounds such as calcitonin, biphosphonates, RANK/RANKL/OPG system and selective estrogen receptor modulators (SERM), (Apostu, D., et al., (2017) Drug Metabolism Reviews, 49:1, 92-104, DOI: 10.1080/03602532.2016.1277737, which is incorporated herein by reference in its entirety) as well as DKK1- and anti-sclerostin antibodies, e.g., a bispecific antibody for dual inhibition of sclerostin and DKK-1, which has been shown to lead to synergistic bone formation (Florio, M., et al., Nat Commun. 2016; 7: 11505, which is incorporated herein by reference in its entirety).

In various embodiments of the provided methods, the implant is a dental implant or an orthopedic implant.

The following examples are presented in order to more fully illustrate certain embodiments of the invention. They should in no way be construed, however, as limiting the broad scope of the invention.

### EXAMPLES

All of the experimental protocols using animals were reviewed and approved by the UCLA Animal Research Committee (ARC #1997-136) and followed the PHS Policy for the Humane Care and Use of Laboratory Animals and the UCLA Animal Care and Use Training Manual guidelines. All of the animals had free access to food and water and were maintained in regular housing with a 12-h light/dark cycle.

### Statistical Analysis

For in vivo and in vitro biological assays, all data were compared by one-way analysis of variance (ANOVA) with multiple sample correction by Holm's Sequential Bonferroni Procedure. All graphs show the mean±SD, otherwise specified.

### Example 1

### Human BMSCs Exposed to Ti Biomaterials Modulated the Circadian Expression Pattern of Clock Genes

### Ti-Biomaterial Surface Modifications

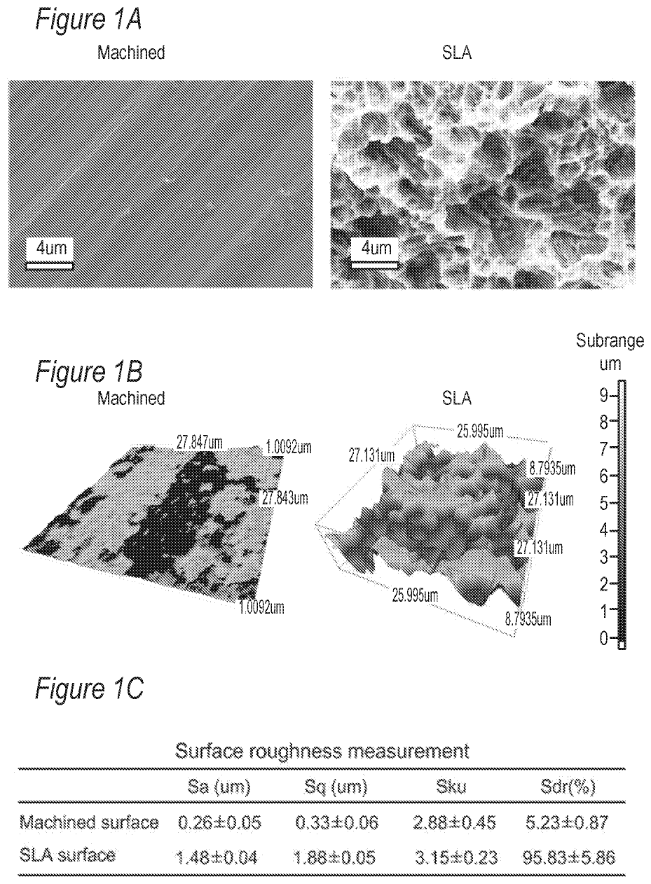

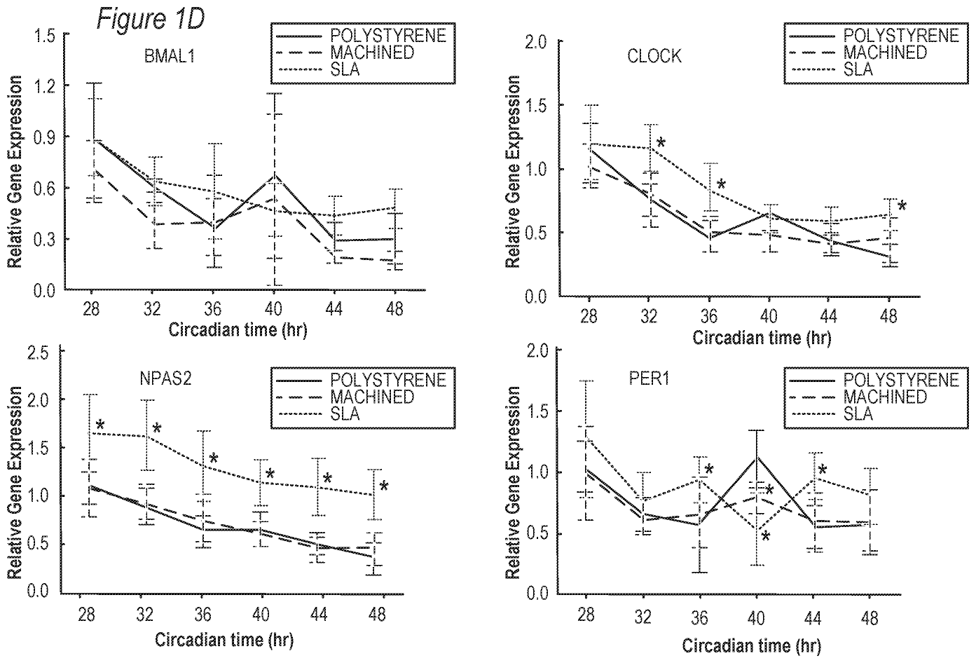

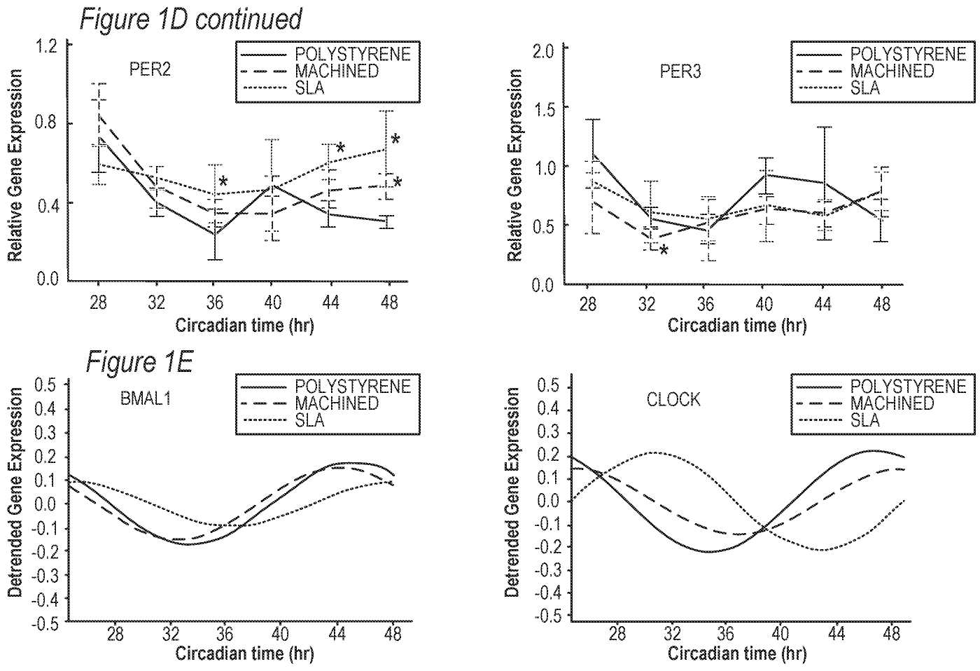

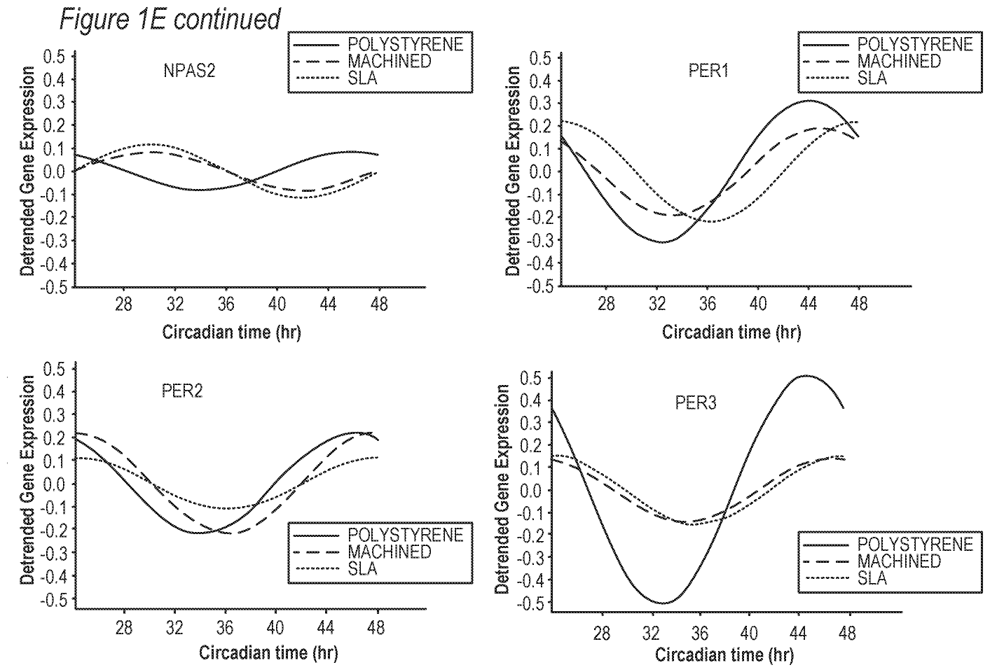

For in vitro studies, commercially pure Ti discs (32 mm in diameter and 1 mm thick) were fabricated. The surface of the Ti discs was treated by sandblasting with large grits and acid etched (SLA) (FIG. 1A) using the production protocols for commercially available dental implants (Neobiotech USA, Inc. Los Angeles, Calif.). Control Ti discs was left as machined. The surface of the Ti discs was characterized by scanning electron microscopy (SEM) and optical interferometric profilometry (n=3 in each group) (FIG. 1B & 1C).

For in vivo studies to evaluate osseointegration in mice, rod-shaped experimental Ti implants (4 mm long and 0.6 mm diameter) were designed to fit to mouse femur bone marrow. The surface of Ti implants were treated with SLA or left as machined. Each Ti implant connected with a handle and was gas sterilized and packaged individually.

### Circadian Rhythm Gene Expression in Human Bone Marrow Stromal Cells (BMSCs)

Immortalized human BMSCs (iMSC3, Applied Biological Materials, Richmond, BC, Canada) were cultured (20,000 cells per cm2) on polystyrene 35-mm culture dishes, machined Ti discs or SLA Ti discs. Starting from 24 hours to 48 hours after the synchronization with 10 μM forskolin, BMSCs from each group were harvested every 4 hours, and total RNA was prepared (n=4 per time point in each group). Taqman-based reverse transcription polymerase chain reaction (RTPCR) was performed using commercially available probes for PER1, PER2, PER3, BMAL1, CLOCK and NPAS2, with GAPDH as an internal control (Life Technologies, Grand Island, N.Y.). The amplitude of each time point (n=6 in each group) was compared by analysis of variance (ANOVA) with post-hoc Tukey HSD test. The circadian rhythm of each clock gene in different culture substrates was analyzed by detrended harmonic regression analysis according to Yang, R., and Su, Z. (2010) Bioinformatics 26, i168-174.

### Results

This study used machined and SLA-treated Ti biomaterials. The surface microtopographic characteristics and measurements (FIGS. 1A-1C) were consistent with published data (Buser, D., et al., (2004) J Dent Res 83, 529-533).

Since the clock genes oscillate with a 24-hour cycle length, the effect of Ti discs on the clock gene expression was tested in every 4 hours for 24 hours in synchronized cells. Forskolin-synchronized human BMSCs maintained on a conventional culture plate demonstrated a circadian expression of PER1, PER2, and PER3 with a peak expression at 36 h to 44 h (FIG. 1D), which was consistent with the inventors' previous observation (Hassan, N., et al., (2017) PLoS One 12, e0183359). When BMSCs were exposed to the SLA disc, there was a striking upregulation of NPAS2 throughout the 24-hour period (FIG. 1D). The detrended harmonious regression analysis revealed that Ti biomaterials significantly altered the circadian expression pattern of all clock genes examined (FIG. 1E). In particular, the SLA disc environment altered the circadian timing of Clock and Npas2, whereas decreased the amplitude of Per genes. The effect of the machined Ti disc was also noted albeit less significant.

The results of this study led us to propose a molecular regulatory mechanism for the establishment of enhanced osseointegration by BMSC exposed to Ti biomaterials with a complex surface. As noted above, human BMSC exposed to Ti disc with SLA treatment demonstrated the significant upregulation of NPAS2 (FIG. 1D), which was highly reproducible with previous data obtained with Ti disc with Nanotite™ treatment, whereas the effect of the Ti disc on other clock genes appeared to be less consistent. It has been reported that Npas2 was not detected in mouse femur BMSC and thus could not be involved in bone remodeling and homeostasis. The present finding suggests that Ti biomaterial-induced Npas2 regulates BMSC to facilitate the implant os seointegration.

### Example 2

### Ti Implant Placed in Mouse Femur Demonstrated Osseointegration

### Experimental Ti Implant Placement in Mouse Femurs

Male 10˜15-wk-old C57Bl/6J mice underwent surgical placement of Ti implants. After anesthesia with isoflurane inhalation, the distal femur was accessed via medial parapatellar arthrotomy with lateral displacement of the quadriceps-patellar complex. After locating the femoral intercondylar notch, the femoral intramedullary canal was manually reamed with a 25-gauge needle for entry into the canal and further reamed with a 23-gauge needle. A Ti implant was inserted in a retrograde fashion into each femur of a mouse. The Ti implant was clipped at 4 mm and further inserted 4 mm using a periodontal probe. The quadriceps-patellar complex was reduced to its anatomic position, and the surgical site was closed using Vicryl 5-0 sutures. Carprofen (5.0 mg/kg) (Rimadyl, Zoetis US, Parsippany, N.J.) was administered subcutaneously at the time of surgery and every 24 hours for 2 days after surgery.

### Implant Push-Out Measurement of Osseointegration

Mice were euthanized and femur bones were harvested at the predetermined healing time. Distal epiphyseal cartilage and the medial half of the femur were removed using a dental diamond disc to locate the implant without overheating. The femurs were then embedded vertically in an acrylic resin block so that the mesial, flat end of the implant was exposed. The mechanical withholding strength was measured by pushing the implant out from the femur bone marrow using a custom-made stainless-steel pushing rod mounted on a 1000-N load cell (Instron, Canton, Mass.). The axial load on the implant was applied at a cross-head speed of 1 mm/min, and displacement of the implant and the load were recorded. The displacement load (N) was used as the implant-push out value.

In the initial study, mice were euthanized 1, 2, 3, 4 and 8 weeks after machined and SLA implant placement (n=3, 4, 4, 4 and 4, respectively) and the time course data were obtained. In the subsequent studies, the femur samples were harvested 3 weeks after implant placement.

### Energy-Dispersive X-Ray Spectroscopy (EDS) and Scanning Electron Microscopy (SEM)

After the implant push-out test, the dislocated Ti implants were recovered from femur bones. The implant surface was scanned by EDS (Supra 40VP SEM, ZEISS, Thornwood, N.Y.). EDS analysis was completed in 5 segments, covering the entire length of the implant. The elemental composition of Ti, calcium (Ca) and phosphorous (P) was determined from the mean of the 5 segment measurements for each implant. The recovered implants were further spatter-coated with iridium (Ir) and examined by SEM (Supra 40VP SEM, ZEISS, Thornwood, N.Y.).

### Nondecalcified Histology of Mouse Femurs Containing Ti Implants

In a separate experiment, femurs were harvested 1, 2, 3 and 4 weeks after Ti implant placement (n=4 at each time point) and fixed in 10% buffered formalin. Femurs were first subjected to microCT scanning (μCT40, Scanco Medical, Wayne, Pa.) and then processed for nondecalcified longitudinal histological sections (Ratliff Histology Consultant, LLC, Franklin, Tenn.). Using microCT image as a guide, a histological section (30 μm) from each specimen was prepared by a grounding system and stained with Goldner trichrome. The bone-implant contact (BIC) ratio was determined over the entire implant periphery.

### Results

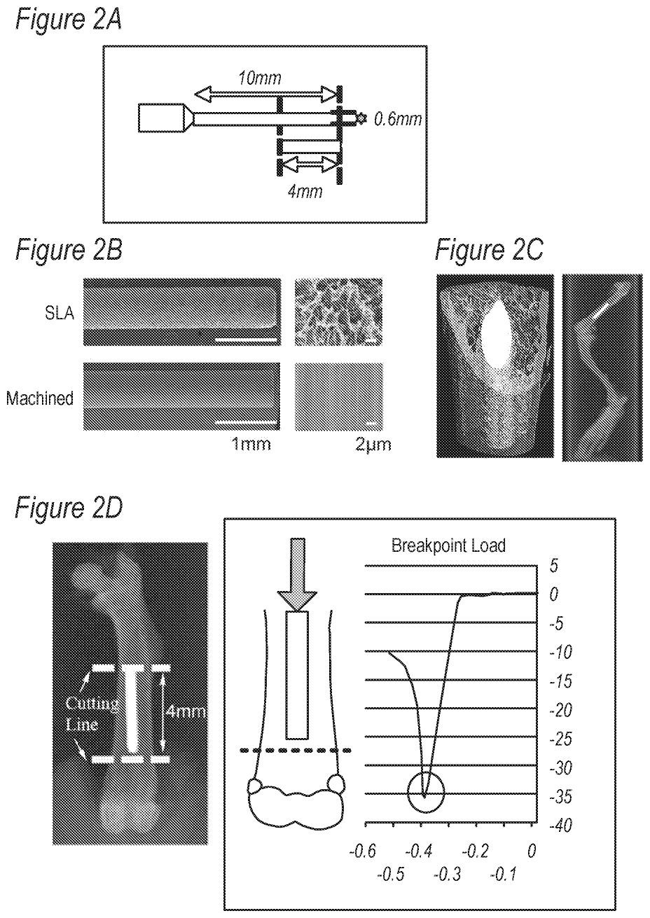

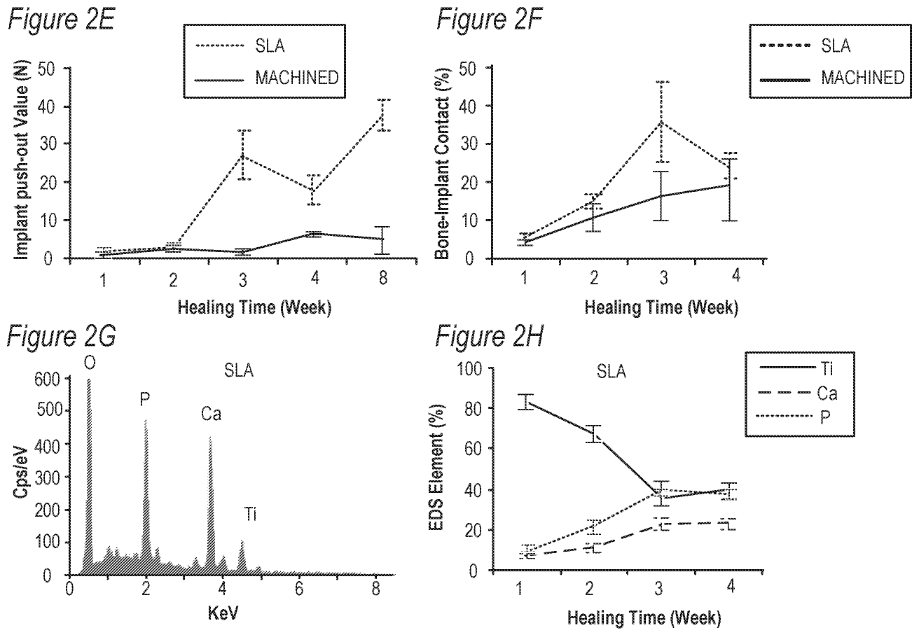

Mouse implant models have been reported, which placed a Ti wire retrograde into the bone marrow space of femurs through epiphyseal cartilage and growth plate, or a small rod or screw was perpendicularly inserted in the middle of femurs. An experimental Ti implant with machined or SLA surface was placed in the distal half of the mouse femur bone marrow without contact with epiphyseal cartilage and growth plate (FIG. 2A-2C). The mechanical withholding shear strength is a hallmark of successful osseointegration. The implant push-out test was designed to measure load at the shear break point in this study (FIG. 2D). The implant push-out test demonstrated the development of mechanical withholding strength in both SLA and machined implants, while the former generated much higher implant push-out values than the latter. The push-out value of the SLA implant increased 3 weeks after implant placement and reached a plateau with a noticeable transient decrease at 4 weeks (FIG. 2E). The BIC ratio of the SLA implant measured in nondecalcified histological sections revealed a progressive increase from 1 week to 3 weeks, followed by a small decrease at 4 weeks (FIG. 2F). There was a slow increase in the BIC ratio of the machined implant. It must be noted that BIC ratios showed significant variations. The BIC ratio at 10%˜15% did not seem to contribute to the mechanical withstanding function under the implant push-out test in this model.

The dislodged Ti implant after the push-out test was subjected to EDS analysis. The implant surface elements were largely Ti, Ca, P and O (FIG. 2G). The weight % of Ti of the recovered SLA implants progressively decreased from 1 week to 3 weeks and reached a plateau. The EDS data of Ti weight % mirrored a reverse trend of BIC. Therefore, the EDS element analysis of the exposed Ti weight %, which showed a clear trend with much less variation, may be a viable surrogate measure for BIC. The weight % of P and Ca increased until 3 weeks (FIG. 2H). Once reaching a plateau at 3 weeks and 4 weeks, the Ca and P measurements on the SLA implant maintained a stable ratio.

### Example 3

### Npas2+/− and Npas2−/− Mice Lack of Femur Bone Abnormalities

### Characterization of Femur Bone of Npas2+/− and Npas2−/− Mice

Npas2+/− mice (24) on the C57Bl/6J background were generated from cryopreserved sperm samples (B6.129S6-Npas2tm1S1m/J, Jackson Laboratory, Bar Harbor, Me.), and an active breeding colony was established at UCLA. Genotype was determined by PCR. Femurs from C57Bl6J wild-type (WT: Npas2+/+), Npas2+/− and Npas2−/− mice were measured for anatomical length and characterized by micro-CT. Femurs were also evaluated by EDS for Ca and P.

### Results

### The Lack of Femur Bone Abnormalities in Npas2+/− and Npas2−/− Mice

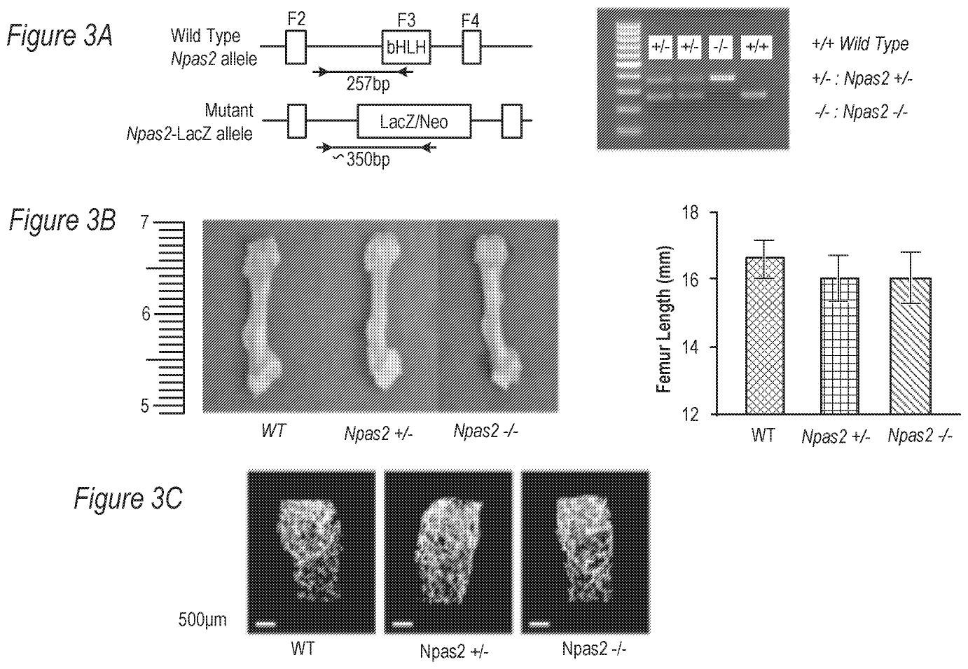

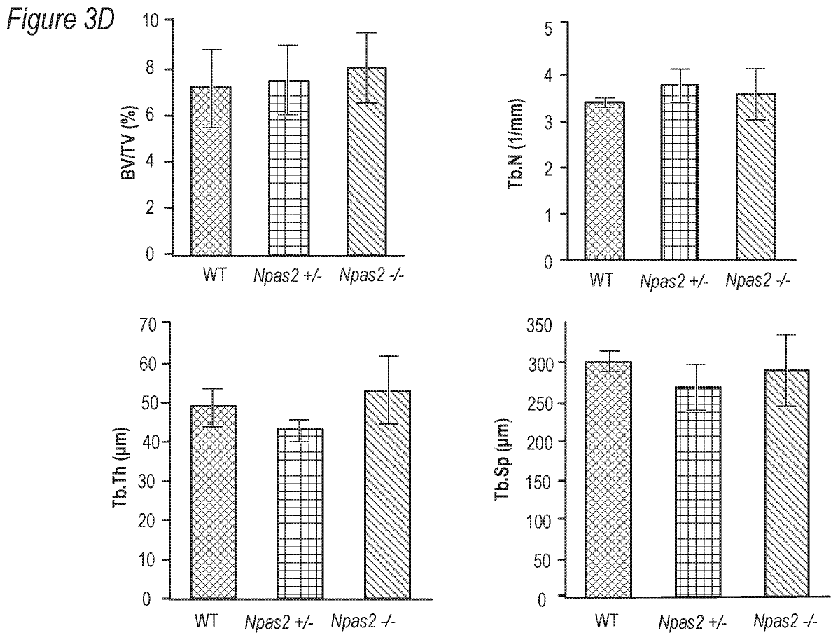

The functional basic helix-loop-helix (bHLH) domain of Npas2 is largely encoded by exon 3, which was replaced by a LacZ/Neo cassette (FIG. 3A), resulting in the synthesis of Npas2 molecules without DNA binding bHLH domains. Femurs harvested from WT, Npas2+/− and Npas2−/− mice were indistinguishable in terms of anatomical size and shape (FIG. 3B) and the interior trabecular bone architecture (FIG. 3C & 3D). Thus, Npas2 KO mice were determined to be a suitable mouse model for investigating the mechanism of implant osseointegration in this study.

### Example 4

### Npas2 KO Mutation Decreased Implant Osseointegration with the SLA Surface but not with the Machined Surface

### Implant Osseointegration in WT, Npas2+/− and Npas2−/− Mice

Male 10˜15-wk-old WT, Npas2+/− and Npas2−/− mice received Ti implants in their femurs, as described. Three (3) weeks after the implant placement, mouse femurs were harvested, and the implant push-out test was conducted. After the implant push-out test, the dislodged Ti implants were carefully recovered from the femur bone marrow and subjected to EDS and SEM analyses. In a separate experiment, 3 weeks after the implant placement, femurs were harvested and processed for nondecalcified longitudinal sectioning of the plastic-embedded femur and implant for histological observation.

### Results

Npas2 KO Mutation Decreased Implant Osseointegration with the SLA Surface but not with the Machined Surface

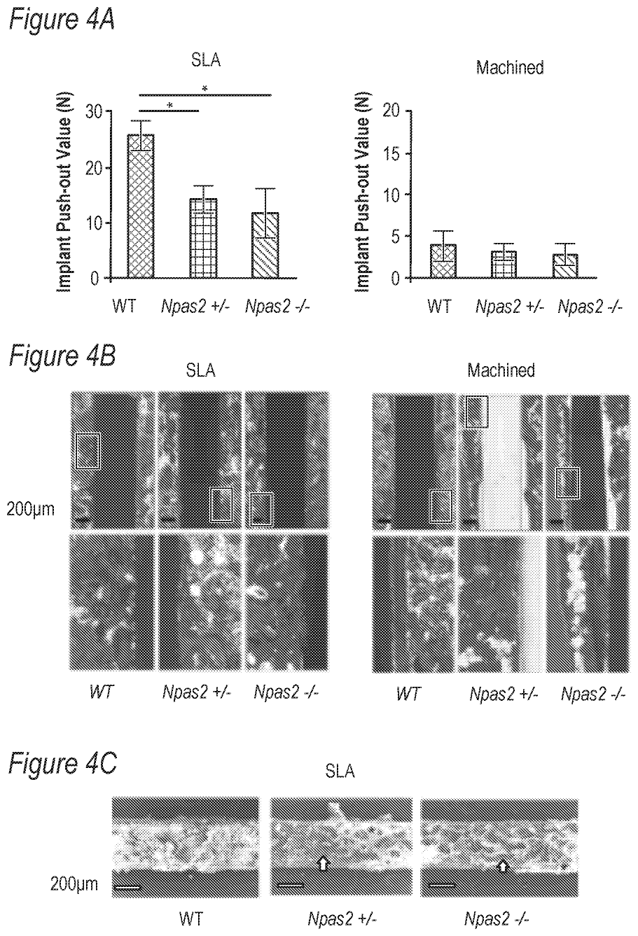

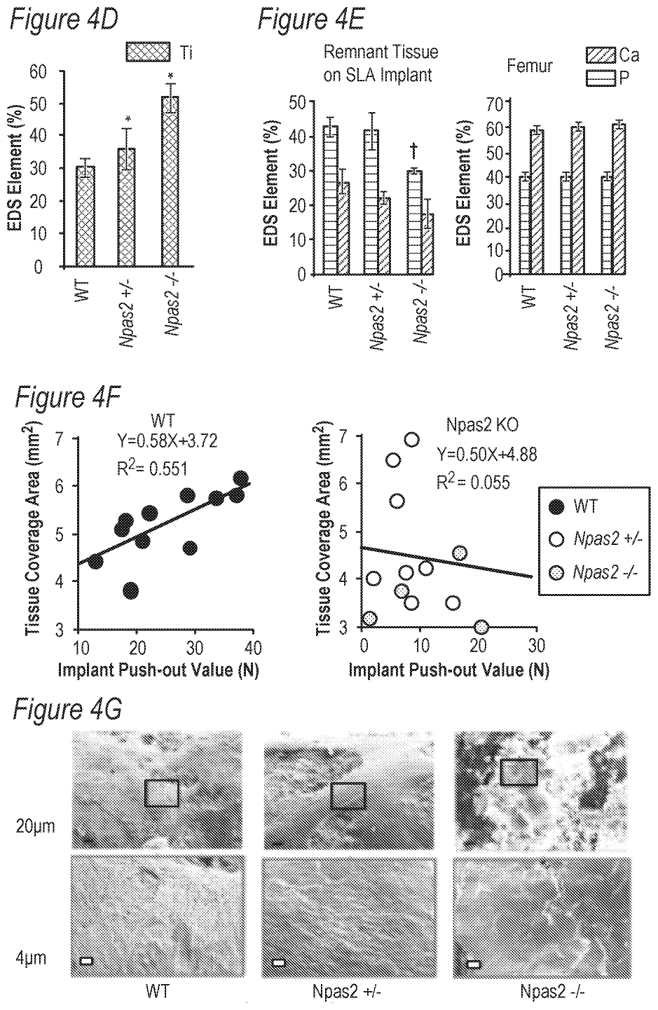

The role of Npas2 on osseointegration after 3 weeks of implant placement in femurs of Npas2 KO mice was examined. The push-out value of the SLA implant was significantly decreased in both Npas2+/− and Npas2−/− mice as compared to WT mice (FIG. 4A). By contrast, the push-out value of the machined implant was not affected by Npas2 KO mutation. Nondecalcified histology revealed the formation of bone tissue around Ti implants in WT and Npas2 KO mice. The bone and implant contact appeared to occur in WT and Npas2 KO mice (FIG. 4B). The experimental implants were recovered after the push-out test and subjected to SEM analysis. The surfaces of the SLA implants recovered from WT mice were widely covered by tissue remnants, whereas those recovered from Npas2+/− and Npas2−/− mice had tissue remnants of a unique reticular structure with numerous punch holes exposing the Ti substrate (FIG. 4C), whereas the machined implants were free from tissue attachment (data not shown).

The EDS analysis revealed the much higher Ti content in implants recovered from Npas2+/− and Npas2−/− mice than in those recovered from WT mice (FIG. 4D). The coverage area by the interface tissue was estimated from the Ti weight % on the entire surface of implant:

2π0.6×4.0×(100−Ti %)/100 (mm2), which was significantly smaller in Npas2+/− and Npas2−/− mice than in WT mice (FIG. 4F). The Ca/P ratio estimated from the EDS analysis (FIG. 4E) did not show the effect of Npas2 KO mutation for both the remnant tissue on SLA implants and femur bone surface.

### Abnormal Tissue Formed on SLA Implant Placed in Naps2+/− and Npas2−/− Mice

Whether the interface tissue coverage affected the implant mechanical withholding strength was then examined. A regression analysis revealed a positive correlation in implants placed in WT mice (R2=0.551, FIG. 4F). By contrast, there was no correlation between the tissue coverage area and the implant push-out value of implants placed in Npas2 KO mice. These data indicate that Npas2 KO mutation affected the interface tissue facilitating the bone and implant bonding. High-magnification SEM images depicted well-developed collagen fibers with a crisscrossed structure in the remnant tissue on the implant recovered from WT mice (FIG. 4G). By contrast, the collagen structure was less visible in tissue remnants on the implants recovered from Npas2+/− and Npas2−/− mice.

In the present study, the refined mouse femur Ti implant model (FIGS. 2A-2C) was applied to Npas2 KO mice. It was found that the Npas2 KO mutation significantly impaired the establishment of osseointegration of SLA implant. Npas2+/− and Npas2−/− mice demonstrated the significant loss implant push-out value (FIG. 4A). Furthermore, while histological bone tissues appeared to be unaffected, EDS-based Ti elemental analysis as a surrogate measurement of BIC suggested the decreased bone and implant contact area in the mutant animals (FIG. 4F). Considering that the expression of Npas2 in BMSC was increased only by the close contact with Ti substrate, a downstream phenotype of Npas2 KO mutation may affect the interface tissue. The remnant tissue on the recovered implant was likely to be the interface tissue between bone and implant, and the interface tissue formed in Npas2+/− and Npas2−/− mice lacked the well-organized collagen fibers (FIG. 4G). Npas2 is a bHLH transcription factor with sequence similarity to a core circadian molecule Clock. However, the lack of its distribution in SCN suggests that Npas2 may be less involved in the maintenance of core circadian rhythm in the central clock but may play more dominant roles in peripheral tissues. Chromatin immunoprecipitation with DNA sequencing (ChIP-Seq) of mouse liver tissue indicated that Npas2-associated target genes were not limited to circadian rhythm-related genes. It has been reported that bHLH transcriptional factors are known to affect BMSC differentiation. Therefore, the inventors speculate that the Ti biomaterial-induced Npas2 may modify the BMSC behaviors suitable for establishing osseointegration.

### Example 5

### Chemical Genetics Analysis Suggested that the Npas2 Upregulation Mechanism Involves Altered Neuroskeletal Pathway

This study further investigated the underlining mechanism of Ti biomaterial-induced Npas2 using a chemical genetics strategy. Chemical genetics is defined as the study of biological systems using small molecule tools, which may be suited to dissect signal transduction pathways responding to the environmental cues.

### Chemical Genetics Analysis

Femur BMSC harvested from Npas2−/− mice were previously characterized for LacZ expression, which was used in this study for high throughput, unbiased screening of Library of Pharmacologically Active Compounds (LOPAC®¹²⁸⁰). (Hassan, N., et al., (2017) PLoS One 12, e0183359). Using 384-well plates, each well was filled with 25 μl non-phenol red DMEM containing 10% FBS and 1% PS, and added 50 nL of LOPAC®¹²⁸⁰ compound (final concentration: 1 μM) using 500 nL pin tool (Biomek FX, Beckman Coulter, Indianapolis, Ind.). To each well, BMSC (Npas2-LacZ) were placed as 1500 cells/25 μl suspension and incubated at room temperature for 1 hour, followed by incubation at 37° C. and 5% CO2 for 48 hours. To measure Npas2-LacZ expression, β-galactosidase activity was measured using a commercially available assay (Beta-Glo Assay System, Promega, Summerville, Calif.).

The Npas2-LacZ expression data were uploaded on an online data analysis tool (CDD Vault, Collaborative Drug Discovery, Inc, Burlingame, Calif.), on which data were normalized and Z-factor was calculated. For this study, hit compounds were selected as Z-score >2.5 or <−2.5.

The selected hit compounds (final concentration: 1 μM) were validated by 3 replicated 384-well plates with mouse BMSC (Npas2-LacZ). The compounds significantly increased or decreased the β-galactosidase activity of BMSC as compared to the untreated cells were selected as candidates. The class, mechanism of action and related functions of the candidate compounds were obtained from the CCD Vault database and literature reviews.

### Expression of Adrenergic Receptors by Human BMSC

Total RNA isolated from human BMSC cultured on polystyrene plate, SLA Ti disc or machined Ti disc as described above was examined for the steady state mRNA levels of α1a, α1b, α1d, α2a, α2b, α2c, β1, β2 and β3 adrenergic receptors using Taqman-based RTPCR.

### Results

### Chemical Genetics Analysis Suggested Npas2 Upregulation Mechanism Involving Altered Neuroskeletal Pathway

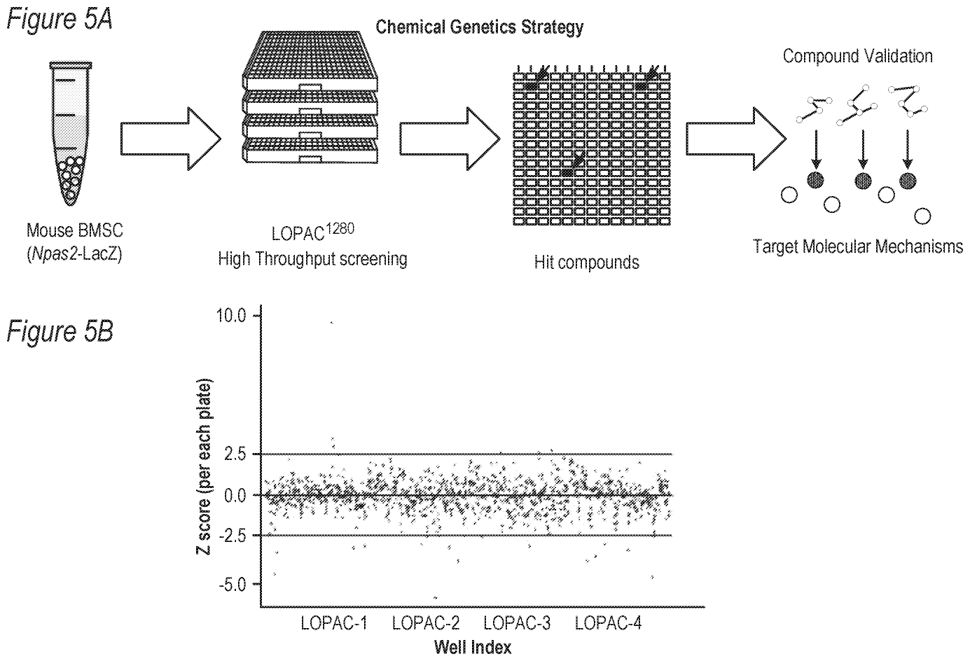

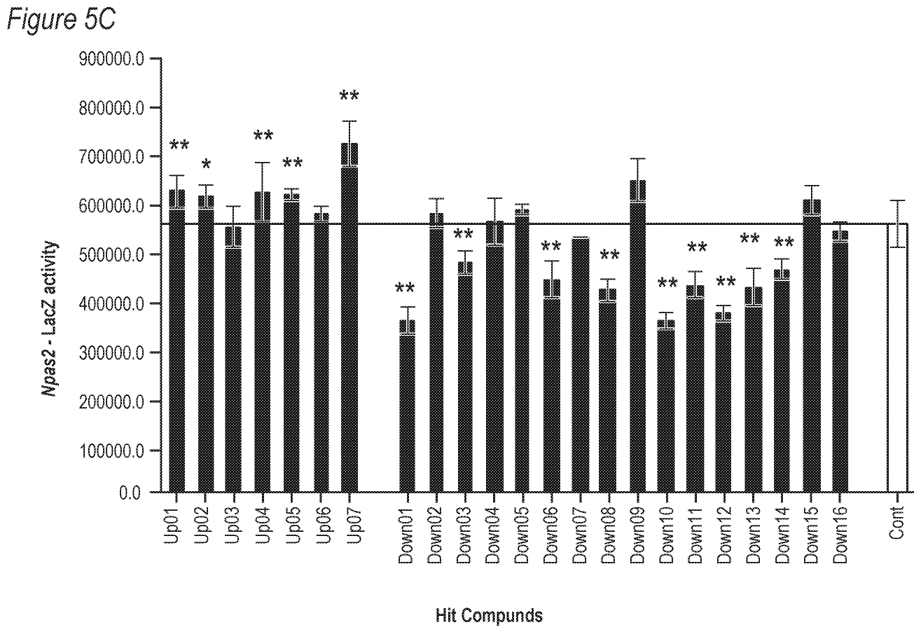

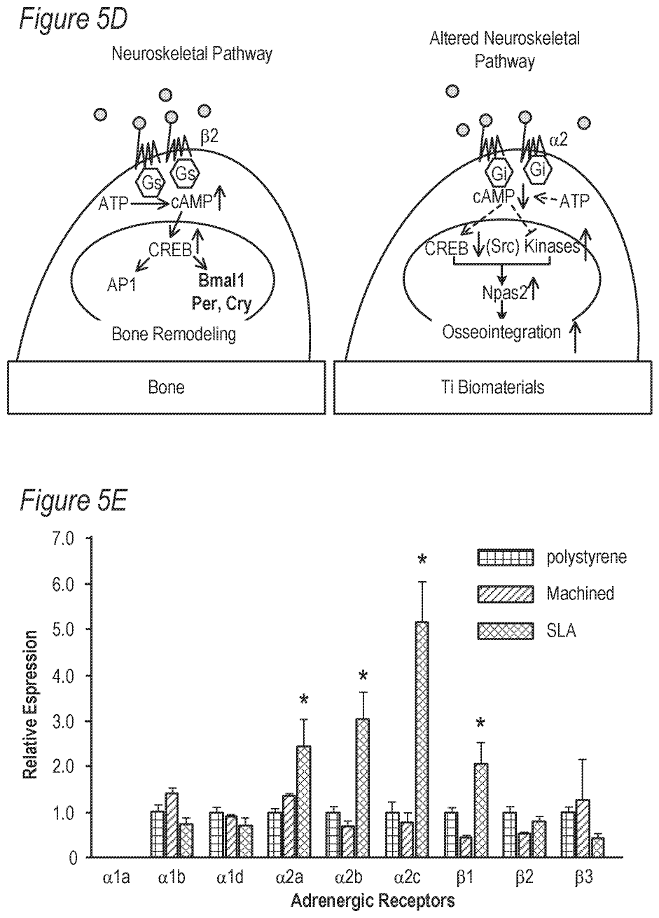

Femur BMSC derived from Npas2−/− mouse was previously characterized for the expression of LacZ (22), which was used for high throughput screening of LOPAC®¹²⁸⁰ (FIG. 5A). The output data of screening analyzed for the Z score >2.5 or <−2.5 resulted in a total of 24 hits: 7 Npas2-upregulation and 16 Npas2-downregulation compounds (FIG. 5B). The validation study identified a total of 14 compounds (FIG. 5C), which were subjected to the chemical genetics analysis. Npas2 upregulating compounds were found to decrease intracellular cAMP or stimulate the α2 adrenergic receptor (Table 1). By contrast, Npas2 down regulating compounds stimulate or accumulate cAMP, or induce cAMP response element binding (CREB) activation.

TABLE 1
Chemical Genetics Analysis using Validated Compounds
Modulating Npas2 Expression

| | | | Description | % |
|---|---|---|---|---|
| | | | and Relevant | Activity |
| Compounds | Class | Action | Functions | * |
| Npas2 | | | | |
| upregulation | | | | |
| 8-(p- | Adenosine | Antagonist | Adenosine | 104.0 |
| Sulfophenyl)- | | | receptor | |
| theophylline | | | antagonist | |
| | | | with selectivity | |
| | | | for A1 over A2 | |
| | | | Blocks/decreases | |
| | | | β adrenergic | |
| | | | receptor- | |
| | | | triggered cAMP | |
| | | | signaling | |
| Psora-4 | K+ Channel | Inhibitor | Potent Kv1.3 | 35.3 |
| | | | potassium | |
| | | | channel | |
| | | | inhibitor | |
| | | | Kv channel | |
| | | | blockers inhibit | |
| | | | cAMP- | |
| | | | stimulated | |
| | | | neuritogenesis | |
| Cilostazol | Cyclic | Inhibitor | Cyclic nucleotide | 26.2 |
| (MS- | Nucleotide | | phosphodiesterase | |
| 1505230) | Phospho- | | catalyzes the | |
| | diesterase | | hydrolysis of | |
| | | | cAMP and cGMP | |
| Methysergide | Serotonin | Antagonist | Semisynthetic | 28.6 |
| maleate | | | ergot alkaloid. | |
| | | | Competitive | |
| | | | □1 adrenergic | |
| | | | receptor blocker | |
| | | | and partial □2 | |
| | | | adrenergic | |
| | | | receptor agonist | |
| Methyldopa | Biochemistry | Agonist | L-aromatic | 18.1 |
| (L, −) | | | amino acid | |
| | | | decarboxylase | |
| | | | inhibitor | |
| | | | α-2 adrenergic | |
| | | | receptor agonist | |
| | | | Decrease | |
| | | | intracellular | |
| | | | cAMP | |
| Harmane | Imidazoline | Agonist | I1 imidazoline | −15.6** |
| (MS-1500866) | | | binding site | |
| | | | agonist | |
| | | | a-2 receptor | |
| | | | agonist | |

* Activity against negative controls in LOPAC ®¹²⁸⁰ screening
**MS-1500866 was initially identified as inhibitor of Npas2, thus it is negative number. However, during the confirmation test, it turned out that it was upregulating Npas2. Thus, this compound was recategorized as inducer.

The effect of sympathetic nervous system on bone remodeling has been extensively investigated as neuroskeletal regulation, in which β2 adrenergic receptor of osteoblasts is thought to play a predominant role (FIG. 5D). The unbiased, chemical genetics analysis suggested that the upregulation of Npas2 could involve the α2 adrenergic receptor. Human BMSC exposed to Ti discs were examined and found to show a robust increase in the expression of α2A, α2B and α2C adrenergic receptors by SLA disc, while β2 adrenergic receptor was not affected (FIG. 5E).

The high throughput screening of pharmacologically active compounds by Npas2-LacZ reporter gene expression resulted in small molecules with known functions in three pathways. The first group clustered in common functions of modulating intracellular cAMP signaling. The compounds increased or reducing cAMP were found reducing or increasing Npas2 expression, respectively (Table 1). The second group was composed of agonists of α2 adrenergic receptors, which increased Npas2. The third group was known to disintegrate cytoskeleton, which might decrease BMSC viability resulting in an artificially low detection of Npas2-LacZ activity. The first and second groups of compound functions suggested the possible involvement of the activation of α2 adrenergic receptors in Ti biomaterials-induced Npas2. In fact, human BMSC exposed to SLA Ti disc exhibited significantly upregulated α2 adrenergic receptors (FIG. 5E). The agonist-activated α2 adrenergic receptors are shown to activate the coupling to G proteins with the highest affinity to inhibitory G protein (Gi). The α2 adrenergic receptors-activated Gi protein decreases adenylyl cyclase activity leading to a decrease in intracellular cAMP.

During the osteogenic differentiation of mouse iPS cells, β2 adrenergic receptor mRNA was found consistently expressed, while α2 adrenergic receptor mRNA was not detected. The activation of β2 adrenergic receptor was involved in osteogenesis of mouse BMSC through cAMP signaling. The effect of neurotransmitters on bone remodeling is known as neuroskeletal regulation, involving predominantly β2 adrenergic receptor of BMSC. α2 adrenergic receptor agonists and β2 adrenergic receptor blockers are commonly used to treat hypertension. A non-selective blocker of β adrenergic receptor was shown to enhance implant osseointegration in rats. A recent retrospective clinical cohort study reported that the use of anti-hypertension medications was associated with a higher dental implant survival.

Contact guidance refers to the phenomenon that cells adjust the orientation and shape following the patterns of the substrate biomaterials. The effect of contact guidance further extends the signal transduction of adherent cells, leading to the differential cell behaviors between the rough and smooth surfaces. However, the complexity in cellular behaviors has been a barrier to determining the pathways of osseointegration. The outcome of this study provided a novel clue to uncovering the molecular and cellular mechanism of accelerated osseointegration. Here the inventors propose that altered neuroskeletal regulatory pathway as a molecular mechanism of BMSC leading to the establishment of enhanced osseointegration. The Ti biomaterial-induced α2 adrenergic receptor expression and Npas2 upregulation may also shed a light on future therapeutic strategies to improve osseointegration or to re-establish implant-bone integration.

### Example 6

### Methyldopa Improves Implant Osseointegration In Vivo

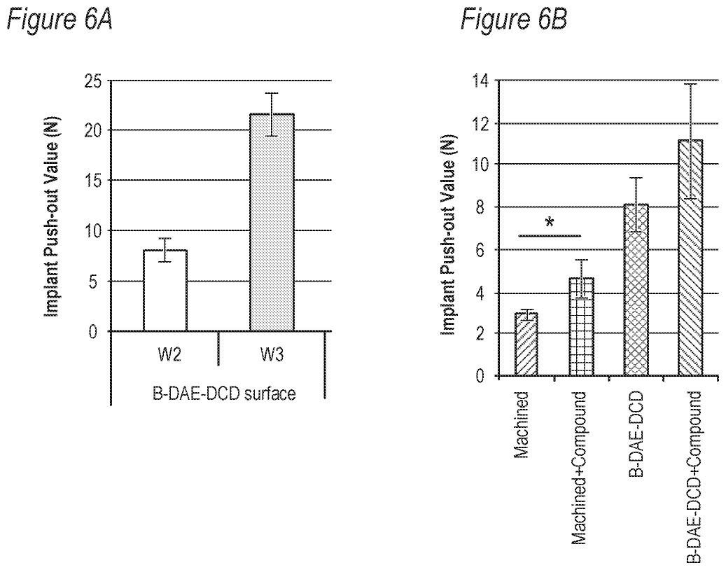

A mouse experimental implant study with B-DAE-DCD surface was tested for the time course of osseointegration using implant push-out test. As shown in FIG. 6A, the push-out value increased from week 2 (W2) and further increased to week 3 (W3). Therefore, the effect of Npas2 upregulating compound (methyldopa) at W2 was tested.

FIG. 6B shows the result of an experimental implant study of machined surface (smooth surface) or B-DAE-DCD surface (rough surface) was placed in the mouse femur. Mice were treated with daily intraperitoneal (IP) injections of methyldopa (L-(−)-a-methyldopa: CAS555-30-06, 75 mg/Kg in 0.9% NaCl solution) or vehicle 0.9% NaCl by IP injections for 2 weeks. Mouse femurs were harvested after 2 weeks of implant surgery and subjected to the implant push out test. Compound (methyldopa) treated mice showed greater push-out value than vehicle treated control mice;*: p<0.05 vs. machined implant without compound.

This in vivo study demonstrates that the Npas2 up regulator compound methyldopa improves implant osseointegration during the early healing stage.

All references, including publications, patent applications, and patents, cited herein are hereby incorporated by reference in their entirety herein.

The examples are presented in order to more fully illustrate embodiments of the invention. They should in no way be construed, however, as limiting the broad scope of the invention.

Having described certain embodiments of the invention with reference to the accompanying drawings, it is to be understood that the invention is not limited to the precise embodiments, and that various changes and modifications may be affected therein by those skilled in the art without departing from the scope or spirit of the invention as defined in the appended claims.

1. A method for enhancing or accelerating osseointegration of an implant into bone marrow of a subject, the method comprising increasing expression of peripheral clock neuronal PAS domain protein 2 (NPAS2) in the bone marrow.

2. The method of claim 1, wherein expression of NPAS2 is increased by administration of a Npas2 modulating compound to the subject.

3. The method of claim 1, wherein the implant comprises titanium, a titanium alloy, chrome or steel.

4. The method of claim 1, wherein the implant comprises a smooth surface and/or a complex surface.

5. The method of claim 2, wherein the Npas2 modulating compound is administered into the bone marrow concurrently with implantation of the implant at an implant location.

6. The method of claim 2, wherein the Npas2 modulating compound is administered into the bone marrow before implantation of the implant.

7. The method of claim 2, wherein the Npas2 modulating compound is administered into the bone marrow after implantation of the implant.

8. The method of claim 2, wherein the Npas2 modulating compound is coated onto a surface of the implant prior to implantation thereof.

9. The method of claim 2, wherein the Npas2 modulating compound upregulates Npas2.

10. The method of claim 9, wherein Npas2 upregulation decreases intracellular cAMP.

11. The method of claim 9, wherein Npas2 upregulation stimulates α2 adrenergic receptor expression.

12. The method of claim 11, wherein the α2 adrenergic receptor is an α2A-, α2B- and/or an α2C-adrenergic receptor.

13. The method of claim 2, wherein the Npas2 modulating compound is an adenosine A1 receptor antagonist, the adenosine A1 receptor antagonist having selectivity for adenosine receptor A1 over adenosine receptor A2.

14. The method of claim 13, wherein the adenosine A1 receptor antagonist is 8-(p-Sulfophenyl) theophylline.

15. The method of claim 13, wherein the adenosine A1 receptor antagonist is selected from the group consisting of 1,3-dipropyl-8-phenylxanthine, 8-(2-amino-4-chlorophenyl)-1,3-dipropylxanthine, 1-isoamyl-3-isobutylxanthine, (R)-3,7-dihydro-8-(1-methyl-2-phenylethyl)-1,3-dipropyl-1H-purine-2,6-dione, (R)-3,7-dihydro-8-(1-phenylpropyl)-1,3-dipropyl-1H-purine-2,6-dione, 1,3-dipropyl-8-cyclopentylxanythine (DPCPX), 8-Cyclopentyl-1,3-dimethylxanthine (CPX), 1,3-dipropyl-8-(3-noradamantyl)xanthine (rolofylline), 1-butyl-3-(3-hydroxypropyl)-8-(3-noradamantyl)xanthine (PSB-36), 1,3-dipropyl-8-[2-(5,6-epoxynorbonyl)]-xanthine (naxifylline), dicyclopropylmethyl (MPDX), 1,3-dipropyl-8-[1-(4-propionate)-bicyclo-[2,2,2]octyl]xanthine (toponafylline), 3-(2-(4-Aminophenyl)ethyl)-8-benzyl-7-(2-(ethyl(2-hydroxyethyl)amino)ethyl)-1-propylxanthine (L-97-1) and analogs or salts thereof.

16. The method of claim 13, wherein the adenosine A1 receptor antagonist is a non-xanthine compound selected from the group consisting of 2-aminothiazole derivatives.

17. The method of claim 2, wherein the Npas2 modulating compound is a Kv1.3 potassium channel inhibitor.

18. The method of claim 17, wherein the Kv1.3 potassium channel inhibitor is 5-(4-phenylbutoxy)psoralen (Psora-4).

19. The method of claim 17, wherein the Kv1.3 potassium channel inhibitor is selected from the group consisting of 5-(3-Phenylpropoxy)psoralen (Psora-3), 5-(5-Phenylpentoxy)psoralen (Psora-5), 5-(4-Biphenylyl)-methoxypsoralen (“Psora-9”) (Psora-9) and 5-(4-phenoxybutoxy)-psoralen (PAP-1).

20. The method of claim 2, wherein the Npas2 modulating compound is an L-aromatic amino acid decarboxylase inhibitor.

21. The method of claim 20, wherein the L-aromatic amino acid decarboxylase inhibitor further is an α2 adrenergic receptor agonist, wherein the compound is L-methyldopa or an analog or derivative thereof.

22. The method of claim 20, wherein the L-aromatic amino acid decarboxylase inhibitor is selected from the group consisting of carbidopa, benserazide α-difluromethyldopa and analogs thereof.

23. The method of claim 2, wherein the Npas2 modulating compound is an imidazoline-1 receptor agonist.

24. The method of claim 23, wherein the imidazoline-1 receptor agonist is harmane.

25. The method of claim 23, wherein the imidazoline-1 receptor agonist is selected from the group consisting of clonidine hydrochloride or clonidine analog, naphazoline hydrochloride, naphazoline hydrochloride, xylometazoline hydrochloride, moxonidine hydrochloride, rilmenidine hemifumarate, a 2-aminothiazoline derivative, and an analog or derivative thereof.

26. The method of claim 25, wherein the 2-aminothiazoline derivative is selected from the group consisting of 2-diethyl-2-aminothiazoline, 2-ethyl-hexylamine-2-aminothiazoline and an analog or derivative thereof.

27. The method of claim 23, wherein the imidazoline-1 receptor agonist is selected from the group consisting of marsanidine, 7-methyl-marsanidine, 7-Cl-marsanidine, 7-F-marsanidine and an analog or derivative thereof.

28. The method of claim 2, wherein the Npas2 modulating compound is a serotonin receptor antagonist selected from the group consisting of methysergide, amesergide and methylergometrine.

29. The method of claim 2, wherein the Npas2 modulating compound is a cyclic nucleotide phosphodiesterase (PDE) inhibitor, wherein the PDE inhibitor is a PDE3 inhibitor.

30. The method of claim 29, wherein the PDE3 inhibitor is selected from the group consisting of cilostazol, a cilostazol analog, milrinone, amrinone, pelrinone, enoximone, pimobendan, meribendan, cilostamide, OPC-33540 and trequinsin.

31. The method of claim 4, wherein the smooth surface implant further comprises a complex surface.

32. The method of claim 31, wherein the complex surface is prepared by sandblasting with large-grit and acid-etching (SLA).

33. The method of any one of claim 1 or 31, wherein Npas2 upregulation occurs in human bone marrow stromal cells (BMSC) exposed to a surface of the implant, wherein the surface is a smooth surface and/or a complex surface.

34. The method of claim 33, wherein the Npas2 upregulation in BMSC facilitates bonding of bone and implant surface at an interface tissue between the bone and the implant.

35. The method of claim 34, wherein the Npas2 upregulation in BMSC stimulates synthesis of dense collagen fibers on the interface tissue, wherein the collagen structure is crisscrossed.

36. The method of claim 35, wherein the Npas2 upregulation in BMSC further stimulates synthesis of dense collagen fibers on the implant surface, wherein the collagen structure is crisscrossed.

37. The method of claim 1, wherein the implant is a dental implant or an orthopedic implant.

38. The method of claim 1, wherein expression of NPAS2 is increased by administering to the subject an adenoviral vector, the adenoviral vector comprising a nucleic acid encoding a human NPAS2 polypeptide.

39. The method of claim 38, wherein the adenoviral vector is administered to the subject at an implant location.

40. The method of claim 38, wherein the adenoviral vector is administered into the bone marrow concurrently with implantation of the implant at the implant location.

41. The method of claim 38, wherein the adenoviral vector is administered into the bone marrow before implantation of the implant.

42. The method of claim 38, wherein the adenoviral vector is administered into the bone marrow after implantation of the implant.

43. A method for accelerating osseointegration of an implant into bone marrow of a subject, the method comprising administering to the subject a pharmaceutical composition comprising an NPAS2 polypeptide or an Npas2 modulating compound, wherein the NPAS2 polypeptide or the Npas2 modulating compound increases expression of peripheral clock neuronal PAS domain protein 2 (NPAS2) in the bone marrow.

44. The method of claim 43, wherein the implant comprises titanium, a titanium alloy, chrome or steel.

45. The method of claim 43, wherein the implant comprises the implant comprises a smooth surface and/or a complex surface.

46. The method of claim 43, wherein the Npas2 modulating compound is an adenosine A1 receptor antagonist having selectivity for adenosine receptor A1 over adenosine receptor A2.

47. The method of claim 46, wherein the adenosine A1 receptor antagonist is 8-(p-Sulfophenyl) theophylline.

48. The method of claim 46, wherein the adenosine A1 receptor antagonist is selected from the group consisting of 1,3-dipropyl-8-phenylxanthine, 8-(2-amino-4-chlorophenyl)-1,3-dipropylxanthine, 1-iso amyl-3-isobutylxanthine, (R)-3,7-dihydro-8-(1-methyl-2-phenylethyl)-1,3-dipropyl-1H-purine-2,6-dione, (R)-3,7-dihydro-8-(1-phenylpropyl)-1,3-dipropyl-1H-purine-2,6-dione, 1,3-dipropyl-8-cyclopentylxanythine (DPCPX), 8-Cyclopentyl-1,3-dimethylxanthine (CPX), 1,3-dipropyl-8-(3-noradamantyl)xanthine (rolofylline), 1-butyl-3-(3-hydroxypropyl)-8-(3-noradamantyl)xanthine (PSB-36), 1,3-dipropyl-8-[2-(5,6-epoxynorbonyl)]-xanthine (naxifylline), dicyclopropylmethyl (MPDX), 1,3-dipropyl-8-[1-(4-propionate)-bicyclo-[2,2,2]octyl]xanthine (toponafylline), 3-(2-(4-Aminophenyl)ethyl)-8-benzyl-7-(2-(ethyl(2-hydroxyethyl)amino)ethyl)-1-propylxanthine (L-97-1) and analogs or salts thereof.

49. The method of claim 46, wherein the adenosine A1 receptor antagonist is a non-xanthine compound selected from the group consisting of 2-aminothiazole derivatives.

50. The method of claim 43, wherein the Npas2 modulating compound is a Kv1.3 potassium channel inhibitor is 5-(4-phenylbutoxy)psoralen (Psora-4).

51. The method of claim 43, wherein the Kv1.3 potassium channel inhibitor is selected from the group consisting of 5-(3-Phenylpropoxy)psoralen (Psora-3), 5-(5-Phenylpentoxy)psoralen (Psora-5), 5-(4-Biphenylyl)-methoxypsoralen (also called “Psora-9”) (Psora-9) and 5-(4-phenoxybutoxy)-psoralen (PAP-1).

52. The method of claim 43, wherein the Npas2 modulating compound is an L-aromatic amino acid decarboxylase inhibitor.

53. The method of claim 52, wherein the L-aromatic amino acid decarboxylase inhibitor further is an α2 adrenergic receptor agonist, wherein the compound is L-methyldopa or an analog or derivative thereof.

54. The method of claim 52, wherein the L-aromatic amino acid decarboxylase inhibitor is selected from the group consisting of carbidopa, benserazide α-difluromethyldopa and analogs thereof.

55. The method of claim 43, wherein the Npas2 modulating compound is an imidazoline-1 receptor agonist.

56. The method of claim 55, wherein the imidazoline-1 receptor agonist is harmane.

57. The method of claim 55, wherein the imidazoline-1 receptor agonist is selected from the group consisting of clonidine hydrochloride or clonidine analog, naphazoline hydrochloride, naphazoline hydrochloride, xylometazoline hydrochloride, moxonidine hydrochloride, rilmenidine hemifumarate, a 2-aminothiazoline derivative, and an analog or derivative thereof.

58. The method of claim 57, wherein the 2-aminothiazoline derivative is selected from the group consisting of 2-diethyl-2-aminothiazoline, 2-ethyl-hexylamine-2-aminothiazoline and an analog or derivative thereof.

59. The method of claim 55, wherein the imidazoline-1 receptor agonist is selected from the group consisting of marsanidine, 7-methyl-marsanidine, 7-Cl-marsanidine, 7-F-marsanidine and an analog or derivative thereof.

60. The method of claim 43, wherein the Npas2 modulating compound is a serotonin receptor antagonist selected from the group consisting of methysergide, amesergide and methylergometrine.

61. The method of claim 43, wherein the Npas2 modulating compound is a cyclic nucleotide phosphodiesterase (PDE) inhibitor, wherein the PDE inhibitor is a PDE3 inhibitor.

62. The method of claim 61, wherein the PDE3 inhibitor is selected from the group consisting of cilostazol, a cilostazol analog, milrinone, amrinone, pelrinone, enoximone, pimobendan, meribendan, cilostamide, OPC-33540 and trequinsin.

63. The method of claim 43, wherein expression of NPAS2 further is increased by administering to the subject an adenoviral vector, the adenoviral vector comprising a nucleic acid encoding a human NPAS2 polypeptide.

64. The method of claim 63, wherein the adenoviral vector is administered to the subject at an implant location.

65. The method of claim 63, wherein the adenoviral vector is administered into the bone marrow concurrently with implantation of the implant at the implant location.

66. The method of claim 63, wherein the adenoviral vector is administered into the bone marrow before implantation of the implant.

67. The method of claim 63, wherein the adenoviral vector is administered into the bone marrow after implantation of the implant.
